SEQUENCE LISTING (56 sequences)

SEQ ID NO: 1 - DNA, 1884 bp, Artificial Sequence
  fcs nucleotide sequence
atgaataacgaagcccgctcagggtcgaccgaccctggccaacgtccgcgctaccgccaggtggccatcgggcatccccaggtgcaggtcagtcacgtcgacgacgtgctgcgcatgcaacctgtcgagccactggcgccgctgccggcgcgcctgctcgagcgcctggtgcattgggcccaggtgcgcccggacaccactttcatcgcggcacgccaggcagacggtgcctggcgttcgatcagctacgtgcagatgctcgccgatgtgcgcaccatcgccgccaacttgctaggactgggcctcagtgccgagcgcccgctggcgctgctttccggcaacgacatcgaacacctgcaaatcgccctcggcgccatgtatgccggtattgcctattgcccggtgtcgccggcctacgcgctgttgtcgcaagacttcgccaagttgcgccatgtctgcgaggtgctcacccccggagtggtcttcgtcagcgacagccagccgttccagcgcgccttcgaggcggtgctggacgattcggtcggcgtgatcagcgtgcgtggccaggtcgcaggtcgcccccatataagcttcgacagcctgttgcaaccgggtgacctggcggcggccgatgcggctttcgccgccaccgggccggacaccatcgccaaattcctcttcacctcgggctcgaccaagctgcccaaggcggtgatcaccacccagcgcatgctgtgcgccaatcagcagatgcttctgcagacttttccgacgttcgccgaggagccgccggtgctggtggactggctgccgtggaaccacacgttcggcggtagccacaacctcggcatcgtgctttacaacgggggcagtttctacctggacgccggcaagccgaccccgcaaggcttcgccgagaccttgcgcaatttgcgcgagatttcccccacggcctacctcaccgtacccaagggctgggaggaactggtcaaggcactggagcaggaccccgcgctacgcgaggtgttctttgcccgcatcaagctgttcttctttgccgccgcaggcctgtcgcaaagcgtctgggaccggctggaccgcattgccgagcaacactgtggcgaacgcatccgcatgatggccggccttggcatgaccgaagcctcgccatcgtgcaccttcaccaccgggcctttgtcgatggccggctatgtcgggctgccggcacctggctgcgaagtgaagctggtgccggtgggcgacaagctcgaggcgcgcttccgtggcccgcatatcatgccgggctactggcgctcgccgcagcagaccgccgaggcgttcgacgaggagggcttctactgttcgggcgacgcgttgaagctggccgatgccaggcagcccgagcttggcctgatgttcgatggccgtatcgctgaggacttcaaactttcgtccggggtattcgtcagtgtcgggccgctgcgcaaccgcgcagtgctggagggctcgccttacgtacaggacatcgtggtcaccgcgccggaccgtgaatgcctgggcctgctggtgttcccgcgtctgcccgagtgtcggcgcctggccgggctggcagaggatgccagcgatgcgcgggtgctggccaacgacaccgtgcgcagttggttcgctgactggctggagcgcttgaaccgcgatgcccaaggcaacgccagccgtatcgaatggctgtcgctgctggccgagccgccgtcgatcgacgccggtgaaatcaccgacaagggctcgatcaatcagcgcgccgtgctgcagcggcgcgccgctcaggtcgaggcgctgtaccgtggcgaagaccccgacgcattgcacgccaaggtgcggccttaa

SEQ ID NO: 2 - protein, 627 aa, Artificial Sequence
  Fcs amino acid sequence
MNNEARSGSTDPGQRPRYRQVAIGHPQVQVSHVDDVLRMQPVEPLAPLPARLLERLVHWAQVRPDTTFIAARQADGAWRSISYVQMLADVRTIAANLLGLGLSAERPLALLSGNDIEHLQIALGAMYAGIAYCPVSPAYALLSQDFAKLRHVCEVLTPGVVFVSDSQPFQRAFEAVLDDSVGVISVRGQVAGRPHISFDSLLQPGDLAAADAAFAATGPDTIAKFLFTSGSTKLPKAVITTQRMLCANQQMLLQTFPTFAEEPPVLVDWLPWNHTFGGSHNLGIVLYNGGSFYLDAGKPTPQGFAETLRNLREISPTAYLTVPKGWEELVKALEQDPALREVFFARIKLFFFAAAGLSQSVWDRLDRIAEQHCGERIRMMAGLGMTEASPSCTFTTGPLSMAGYVGLPAPGCEVKLVPVGDKLEARFRGPHIMPGYWRSPQQTAEAFDEEGFYCSGDALKLADARQPELGLMFDGRIAEDFKLSSGVFVSVGPLRNRAVLEGSPYVQDIVVTAPDRECLGLLVFPRLPECRRLAGLAEDASDARVLANDTVRSWFADWLERLNRDAQGNASRIEWLSLLAEPPSIDAGEITDKGSINQRAVLQRRAAQVEALYRGEDPDALHAKVRP

SEQ ID NO: 3 - DNA, 831 bp, Artificial Sequence
  ech nucleotide sequence
atgagcaaatacgaaggccgctggaccaccgtgaaggtcgaactggaagcgggcatcgcctgggtgaccctcaatcgcccggaaaaacgcaatgccatgagccccaccctgaaccgggaaatggtcgacgtgctggaaacccttgagcaggacgctgacgctggcgtgctggtattgaccggtgccggcgagtcctggaccgccggcatggacctgaaggagtacttccgcgaggtggacgccggcccggaaatcctccaggaaaagattcgtcgcgaagcctcgcaatggcaatggaagttgctgcgtctgtatgccaaaccgaccatcgccatggtcaacggctggtgcttcggcggcggcttcagcccactggtggcatgcgacctggcgatctgcgccaacgaagcgaccttcggcctgtcggaaatcaactggggcatcccgcctggtaacctggtcagcaaggccatggccgataccgttggccatcgtcagtcgctgtactacatcatgaccggcaagaccttcgatggtcgcaaggctgccgagatgggcctggtgaacgacagtgtgccgctggccgagctgcgtgaaaccacccgcgagttggcgctgaacctgctggaaaagaacccggtggtgctgcgtgccgcgaagaatggcttcaagcgttgccgcgagctgacctgggaacagaacgaggactacctctacgccaagctcgaccagtcgcgcctgctggacactaccggcggccgcgagcagggcatgaagcagttcctcgacgacaagagcatcaagccaggcctgcaggcctacaagcgctaa

SEQ ID NO: 4 - protein, 276 aa, Artificial Sequence
  Ech amino acid sequence
MSKYEGRWTTVKVELEAGIAWVTLNRPEKRNAMSPTLNREMVDVLETLEQDADAGVLVLTGAGESWTAGMDLKEYFREVDAGPEILQEKIRREASQWQWKLLRLYAKPTIAMVNGWCFGGGFSPLVACDLAICANEATFGLSEINWGIPPGNLVSKAMADTVGHRQSLYYIMTGKTFDGRKAAEMGLVNDSVPLAELRETTRELALNLLEKNPVVLRAAKNGFKRCRELTWEQNEDYLYAKLDQSRLLDTTGGREQGMKQFLDDKSIKPGLQAYKR

SEQ ID NO: 5 - DNA, 1449 bp, Artificial Sequence
  vdh nucleotide sequence
atgttgcaggtgcctttgctgattggcgggcagtcgcgccccgccagcgatggacgaaccttcgagcgctgtaacccggtgactggcgaggtggtgtcgcaggctgccgccgccacactggccgatgccgatgccgcggtggctgctgccagcgcggcgtttccggcctgggccgccctggcaccgggcgagcggcgcagccgcttgctggcaggcgctgatctgttgcaggcgagggccgccgagttcatcgccgccgccggtgaaaccggggccatggccaactggtatggcttcaacgtgaagttggccgccaacatgctgcgcgaggctgcagccatgaccacgcagatcaccggtgaagtgatcccctcggacgttcccggcagcttcgcaatggccctgcgcgcgccctgcggcgtggtgttgggcatcgcaccgtggaacgccccggtgatactggccacgcgtgccattgccatgccgctggcctgcggcaacaccgtggtgctcaaggcctcggagctgagcccggcggtccatcggctgatcggccaggtgctgcacgatgcaggcatcggcgacggcgtggtcaatgtcatcagcaatgcgccgcaggatgcccccgccatcgtcgagcggctgatcgccaaccctgcggtacgccgggtcaacttcaccggttcgacgcacgtcgggcgcatcgtcggcgaactggcggcccgccatctcaagccggccctgctcgaactgggcggcaaggcacctttgctggtgctcgacgatgccgacctggacgccacggtcgaagcggcggccttcggtgcctacttcaaccaggggcaaatctgcatgtccaccgagcgccttgtggtggacagctgtattgccgacgctttcgtcgacaagctggcggtgaagatcgccgggctgcgtgcaggtgatccgcaagccagcacctcggtgctcggctcgctggtcagcgcagcggccggcgagcgcatcaaggcactgatcgacgatgccgtggccaagggcgcgcgcctggtcagcggcggccagctggaaggcagcatcctgcaaccgaccttgctcgacaacgtcgatgccagcatgcgcctgtaccgcgaggagtccttcggcccggtggcggtggtactgcgcgccgaaggcgacgaagccttgctgcagctggccaacgactcggagttcggtctgtcatcggccattttcagccgcgacaccagccgcgccctggccttggcccaacgggtggagtcgggtatctgccatatcaacggcccgaccgttcacgatgaagcgcagatgccgtttggcggggtcaagtccagcggctatggcagcttcggcagccgcacggccatcgatcagttcacccagttgcgctgggtcaccctccagcacggcccgcgtcactatcccatctaa

SEQ ID NO: 6 - protein, 482 aa, Artificial Sequence
  Vdh amino acid sequence
MLQVPLLIGGQSRPASDGRTFERCNPVTGEVVSQAAAATLADADAAVAAASAAFPAWAALAPGERRSRLLAGADLLQARAAEFIAAAGETGAMANWYGFNVKLAANMLREAAAMTTQITGEVIPSDVPGSFAMALRAPCGVVLGIAPWNAPVILATRAIAMPLACGNTVVLKASELSPAVHRLIGQVLHDAGIGDGVVNVISNAPQDAPAIVERLIANPAVRRVNFTGSTHVGRIVGELAARHLKPALLELGGKAPLLVLDDADLDATVEAAAFGAYFNQGQICMSTERLVVDSCIADAFVDKLAVKIAGLRAGDPQASTSVLGSLVSAAAGERIKALIDDAVAKGARLVSGGQLEGSILQPTLLDNVDASMRLYREESFGPVAVVLRAEGDEALLQLANDSEFGLSSAIFSRDTSRALALAQRVESGICHINGPTVHDEAQMPFGGVKSSGYGSFGSRTAIDQFTQLRWVTLQHGPRHYPI

SEQ ID NO: 7 - DNA, 1068 bp, Artificial Sequence
  vanA nucleotide sequence
atgtaccccaaaaacacctggtacgtcgcctgcacccccgatgagatcgccaccaaacccctgggccggcaaatctgcggggaaaaaatcgtgttctaccgcgcccgcgagaaccaagtagccgccgtcgaggacttctgcccgcaccgcggcgcaccgttgtcgttgggctatgtcgaggacggcaacctggtgtgcggctaccacggcctggtgatgggttgcgacggcaagaccgtgtcgatgccgggccaacgggtgcgtggcttcccctgcaacaagacctttgcggccgtcgagcgctatggcttcatctgggtctggcccggtgaccaggcgcaggccgacccggcgctgattccgcatctggaatgggcggtgagtgatgagtgggcctacggcggcgggctgttccacatcggttgcgactaccgcctgatgatcgacaacctcatggacctcacccatgaaacctatgtgcacgcctccagcatcggccagaaggagatcgacgaggcaccgccggtcaccaccgtcaccggcgacgaagtggtcaccgcccggcacatggaaaacatcatggcgccaccgttctggcgcatggccttgcgtggcaatggcctggccgacgatgtaccagtggaccgctggcaaatctgccgtttcaccccacctagccatgtgctgatcgaagtgggtgtagcgcatgccggcaagggcggctaccacgccgaggcacagcataaggcgtcgagcatcgtggtcgacttcatcacccctgagagcgatacctctatctggtacttctggggcatggcgcgcaacttcgctgcgcacgaccagaccctgaccgacaacattcgtgagggccagggcaagattttcagcgaagacctggaaatgctcgaacgccagcagcagaacctgctggcccaccccgagcgcaacttgctgaagctgaatatcgacgccggcggcgtgcagtcacgcaaagtgctggagcggatcatcgcccaagagcgtgcgccgcagccgcaactgatcgccaccagcgccaaccctgcctga

SEQ ID NO: 8 - protein, 355 aa, Artificial Sequence
  VanA amino acid sequence
MYPKNTWYVACTPDEIATKPLGRQICGEKIVFYRARENQVAAVEDFCPHRGAPLSLGYVEDGNLVCGYHGLVMGCDGKTVSMPGQRVRGFPCNKTFAAVERYGFIWVWPGDQAQADPALIPHLEWAVSDEWAYGGGLFHIGCDYRLMIDNLMDLTHETYVHASSIGQKEIDEAPPVTTVTGDEVVTARHMENIMAPPFWRMALRGNGLADDVPVDRWQICRFTPPSHVLIEVGVAHAGKGGYHAEAQHKASSIVVDFITPESDTSIWYFWGMARNFAAHDQTLTDNIREGQGKIFSEDLEMLERQQQNLLAHPERNLLKLNIDAGGVQSRKVLERIIAQERAPQPQLIATSANPA

SEQ ID NO: 9 - DNA, 951 bp, Artificial Sequence
  vanB nucleotide sequence
atgatcgatgccgtagtggtatcccgtaacgatgaagcgcagggtatctgcagcttcgagctggccgcggcagatggcagcctgctgccggcgttcagcgccggcgcccatatcgacgtgcacctgcccgacgggctggtgcgccagtattcgctgtgcaaccaccccgaagaacgccatcgctatctgattggcgtactcaacgacccggcttcgcggggcggttctcgtagcctgcacgaacaggtgcaagccggtgcccggctgcgtatcagtgcgccgcgcaacctgttcccgctggccgagggtgcgcagcgcagtttgctgtttgctggcggtatcggcattaccccaatcctgtgcatggccgagcagctgtccgacagcggccaggccttcgagctgcactactgtgcccgctccagcgagcgtgcggcgtttgtcgagcgcatccgcagcgcgccgttcgctgatcggctgttcgtgcattttgacgagcagccggaaacggcgctggacatcgcccaggtgctgggcaacccgcaagatgatgtgcacctgtatgtatgcgggcccggcgggttcatgcagcatgtgctggacagcgcgaaggggctgggctggcaggaggccaacctgcaccgcgagtacttcgccgcagcaccggtggatgccagcaacgatggcagtttcgcggtgcaggtgggcagcacgggacaggtgttcgaggtgccagccgaccggaccgtggtgcaggtgctggaagagaatggtatcgagatcgccatgtcgtgcgagcagggtatttgcggcacctgcctgacacgcgtgctgcagggcacaccggaccatcgcgatctgtttctcaccgaagaggaacaggccctgaacgatcagttcacgccctgctgctcgcgctcgaagacgccgctgctggtgctggacatctga

SEQ ID NO: 10 - protein, 316 aa, Artificial Sequence
  VanB amino acid sequence
MIDAVVVSRNDEAQGICSFELAAADGSLLPAFSAGAHIDVHLPDGLVRQYSLCNHPEERHRYLIGVLNDPASRGGSRSLHEQVQAGARLRISAPRNLFPLAEGAQRSLLFAGGIGITPILCMAEQLSDSGQAFELHYCARSSERAAFVERIRSAPFADRLFVHFDEQPETALDIAQVLGNPQDDVHLYVCGPGGFMQHVLDSAKGLGWQEANLHREYFAAAPVDASNDGSFAVQVGSTGQVFEVPADRTVVQVLEENGIEIAMSCEQGICGTCLTRVLQGTPDHRDLFLTEEEQALNDQFTPCCSRSKTPLLVLDI

SEQ ID NO: 11 - DNA, 1188 bp, Artificial Sequence
  pobA nucleotide sequence
atgaaaactcaggttgcaattattggtgcaggtccgtctggcctgctgctgggccagctgctgcacaaggccggtatcgataacatcatcgtcgaacgccagactgccgagtacgtactaggccgcatccgcgccggggtgctagagcaaggcacggtcgacctgctgcgcgaggctggcgtggccgagcgcatggaccgtgaaggcctggtgcacgagggggttgaactgctggttggcgggcgccgccagcgtctggatctcaaagccctgaccggcggcaagacggtgatggtctacggccagaccgaagtcacccgtgacctgatgcaggcccgcgaagccagtggtgcgccgatcatttattcagccgccaacgttcagccgcatgaattgaaaggcgagaagccctacctgacgttcgaaaaggatggccgggtgcagcggattgactgcgactatatcgccggctgcgacggcttccacggtatctcgcggcagagcatcccggagggcgtgctgaaacagtatgagcgggtttacccgtttggctggctgggcctgctgtcggacacaccgccagtcaatcacgagttgatctacgcccaccatgagcgcggtttcgcgttgtgtagccaacgctcgcaaacacgcagccgctactacctgcaggtacctttgcaggatcgggtcgaggagtggtctgacgagcgtttctgggacgaactgaaagcccgtctgcccgccgaggtggcggcggacctggtcacaggccccgcgttggaaaaaagtattgcgccgctgcgtagcctggtggtcgaacccatgcagtatggtcacctgttcctggtgggggacgcggcgcacatcgtcccccctacgggtgccaaaggccttaacctggcggcctccgacgtcaactacctgtaccgcattctggtcaaggtgtaccacgaagggcgcgtcgacctgcttgcgcaatactcgccgctggcactgcgccgcgtgtggaagggcgagcgcttcagctggttcatgacccaactgctgcatgacttcggtagccacaaggacgcctgggaccagaagatgcaggaagctgaccgcgagtacttcctgacctcgccggcgggcctggtgaacattgccgagaactatgtggggctgccgttcgaggaagttgcctga

SEQ ID NO: 12 - protein, 395 aa, Artificial Sequence
  PobA amino acid sequence
MKTQVAIIGAGPSGLLLGQLLHKAGIDNIIVERQTAEYVLGRIRAGVLEQGTVDLLREAGVAERMDREGLVHEGVELLVGGRRQRLDLKALTGGKTVMVYGQTEVTRDLMQAREASGAPIIYSAANVQPHELKGEKPYLTFEKDGRVQRIDCDYIAGCDGFHGISRQSIPEGVLKQYERVYPFGWLGLLSDTPPVNHELIYAHHERGFALCSQRSQTRSRYYLQVPLQDRVEEWSDERFWDELKARLPAEVAADLVTGPALEKSIAPLRSLVVEPMQYGHLFLVGDAAHIVPPTGAKGLNLAASDVNYLYRILVKVYHEGRVDLLAQYSPLALRRVWKGERFSWFMTQLLHDFGSHKDAWDQKMQEADREYFLTSPAGLVNIAENYVGLPFEEVA

SEQ ID NO: 13 - DNA, 720 bp, Artificial Sequence
  pcaH nucleotide sequence
atgcccgcccaggacaacagccgcttcgtgatccgtgatcgcaactggcaccctaaagcccttacgcctgactacaagacctccgttgcccgctcgccgcgccaggcactggtcagcattccgcagtcgatcagcgaaaccactggtccggacttttcccatctgggcttcggcgcccacgaccatgacctgctgctgaacttcaataacggtggcctgcccattggcgagcgcatcatcgtcgccggccgtgtcgtcgaccagtacggcaagcctgtgccgaacactttggtggagatgtggcaagccaacgccggcggccgctatcgccacaagaacgatcgctacctggcgcccctggacccgaacttcggtggtgttgggcggtgtctgaccgaccgtgacggctattacagcttccgcaccatcaagccgggcccgtacccatggcgcaacggcccgaacgactggcgcccggcgcatatccacttcgccatcagcggcccatcgatcgccaccaagctgatcacccagttgtacttcgaaggtgacccgctgatcccgatgtgcccgatcgtcaagtcgatcgccaacccgcaagccgtgcagcagttgatcgccaagctcgacatgagcaacgccaacccgatggactgcctggcctaccgctttgacatcgtgctgcgcggccagcgcaagacccacttcgaaaactgctga

SEQ ID NO: 14 - protein, 239 aa, Artificial Sequence
  PcaH amino acid sequence
MPAQDNSRFVIRDRNWHPKALTPDYKTSVARSPRQALVSIPQSISETTGPDFSHLGFGAHDHDLLLNFNNGGLPIGERIIVAGRVVDQYGKPVPNTLVEMWQANAGGRYRHKNDRYLAPLDPNFGGVGRCLTDRDGYYSFRTIKPGPYPWRNGPNDWRPAHIHFAISGPSIATKLITQLYFEGDPLIPMCPIVKSIANPQAVQQLIAKLDMSNANPMDCLAYRFDIVLRGQRKTHFENC

SEQ ID NO: 15 - DNA, 606 bp, Artificial Sequence
  pcaG nucleotide sequence
atgccaatcgaactgctgccggaaaccccttcgcagactgccggcccctacgtgcacatcggcctggccctggaagccgccggcaacccgacccgcgaccaggaaatctggaactgcctggccaagccagacgccccgggcgagcacattctgctgatcggccacgtatatgacggaaacggccacctggtgcgcgactcgttcctggaagtgtggcaggccgacgccaacggtgagtaccaggatgcctacaacctggaaaacgccttcaacagctttggccgcacggctaccaccttcgatgccggtgagtggacgctgcaaacggtcaagccgggtgtggtgaacaacgctgctggcgtgccgatggcgccgcacatcaacatcagcctgtttgcccgtggcatcaacatccacctgcacacgcgcctgtatttcgatgatgaggcccaggccaatgccaagtgcccggtgctcaacctgatcgagcagccgcagcggcgtgaaaccttgattgccaagcgttgcgaagtggatgggaagacggcgtaccgctttgatatccgcattcagggggaaggggagaccgtcttcttcgacttctga

SEQ ID NO: 16 - protein, 201 aa, Artificial Sequence
  PcaG amino acid sequence
MPIELLPETPSQTAGPYVHIGLALEAAGNPTRDQEIWNCLAKPDAPGEHILLIGHVYDGNGHLVRDSFLEVWQADANGEYQDAYNLENAFNSFGRTATTFDAGEWTLQTVKPGVVNNAAGVPMAPHINISLFARGINIHLHTRLYFDDEAQANAKCPVLNLIEQPQRRETLIAKRCEVDGKTAYRFDIRIQGEGETVFFDF

SEQ ID NO: 17 - DNA, 1353 bp, Artificial Sequence
  pcaB nucleotide sequence
atgagcaaccaactgttcgacgcctatttcaccgcgccggccatgcgcgagattttctccgaccgaggccgcctgcagggcatgctggatttcgaagccgcgcttgcccgagccgaagcctctgccggtttggtcccgcacagcgcggtagcggccatcgaggcggcatgccaggccgagcgctatgacgttggcgcgctggccaatgccatcgccaccgcgggcaactcggccattccgctggtgaaagcgttgggcaaggtgatcgccaccggcgtgccagaggctgagcgctatgtgcaccttggggccaccagccaggatgcgatggataccggtctggttctgcagctgcgcgatgccctcgatttgatcgaggccgacctcggcaagctggccgataccctgtcgcagcaggccttgaagcacgccgatacgcccttggtgggtcgtacctggttgcaacacgccaccccggtgaccctgggcatgaaactggccggtgtactgggtgctttgacccgccaccgtcagcgcctgcaggaactgcgcccgcgccttctggtcctgcagttcggcggtgcctcgggcagcctggcggcgctgggcagcaaggcgatgccggtggccgaagcgctggccgaacagctcaagctgaccctgcccgagcagccctggcacacccagcgcgaccgcctggtggagtttgcctcggtattgggcctggttgccggcagcctgggcaagttcggccgtgatatcagcttgctgatgcaaaccgaggcgggggaggtgtttgagccttctgcgccgggcaagggtggttcttcgaccatgccacacaagcgcaacccggtgggtgccgccgtgttgatcggtgccgcgacccgcgtgccgggcctgctgtcgacgctgttcgcagccatgcctcaggagcacgaacgcagcctgggcctatggcatgccgagtgggaaaccctgccggatatctgctgcctggtctctggcgccctgcgccaggctcaagtgattgccgagggcatggaggtggatgccgcgcgcatgcgccgtaacctcgacctgacccaaggcctggtgctggccgaagcggtgagcatcgtcctcgcccagcgtctgggtcgcgaccgtgcccaccacctgctggaacaatgctgccaacgcgcggtggccgaacagcggcacctgcgtgccgtgctgggtgacgagccgcaggtcagcgccgagctgtctggcgaagaactcgatcgcctgctcgaccctgcccattacctgggccaggcccgcgtctgggtggcgcgcgccgtgtccgaacatcaacgtttcactgcctga

SEQ ID NO: 18 - protein, 450 aa, Artificial Sequence
  PcaB amino acid sequence
MSNQLFDAYFTAPAMREIFSDRGRLQGMLDFEAALARAEASAGLVPHSAVAAIEAACQAERYDVGALANAIATAGNSAIPLVKALGKVIATGVPEAERYVHLGATSQDAMDTGLVLQLRDALDLIEADLGKLADTLSQQALKHADTPLVGRTWLQHATPVTLGMKLAGVLGALTRHRQRLQELRPRLLVLQFGGASGSLAALGSKAMPVAEALAEQLKLTLPEQPWHTQRDRLVEFASVLGLVAGSLGKFGRDISLLMQTEAGEVFEPSAPGKGGSSTMPHKRNPVGAAVLIGAATRVPGLLSTLFAAMPQEHERSLGLWHAEWETLPDICCLVSGALRQAQVIAEGMEVDAARMRRNLDLTQGLVLAEAVSIVLAQRLGRDRAHHLLEQCCQRAVAEQRHLRAVLGDEPQVSAELSGEELDRLLDPAHYLGQARVWVARAVSEHQRFTA

SEQ ID NO: 19 - DNA, 393 bp, Artificial Sequence
  pcaC nucleotide sequence
atggacgagaaacaacgttacgacgctggcatgcaagtgcgccgcgcagtgctgggtgatgcccacgtggaccgcagcctggagaagctcaacgacttcaatggcgagttccaggaaatgatcacccgccacgcctggggtgacatctggacccgcccggggctgccgcgccatacccgcagcctgatcaccatcgccatgctgattggcatgaaccgcaacgacgagctgaagctgcacctgcgtgcggcggccaacaatggcgtgacccgcgacgagatcaaggaagtgctgatgcagagcgcgatctactgcggcattccggcggccaatgccacgttccacctggctgagtcggtgtgggatgaacttggcgtagagtctcgccagtaa

SEQ ID NO: 20 - protein, 130 aa, Artificial Sequence
  PcaC amino acid sequence
MDEKQRYDAGMQVRRAVLGDAHVDRSLEKLNDFNGEFQEMITRHAWGDIWTRPGLPRHTRSLITIAMLIGMNRNDELKLHLRAAANNGVTRDEIKEVLMQSAIYCGIPAANATFHLAESVWDELGVESRQ

SEQ ID NO: 21 - DNA, 792 bp, Artificial Sequence
  pcaD nucleotide sequence
atggcgcacttgcaactggccgatggcgttttgaattaccagatcgatggcccggatgacgccccggtgctggtcctgtccaactcgctgggtaccgacctgggcatgtgggacacccagattccgctctggagtcagcacttccgggtgctgcgctatgacacccgtggtcacggcgcatcgctggtcactgaaggcccttacagcatcgaacagctgggccgcgacgtgctggccctgctcgatggcctggacattcaaaaggctcacttcgtcggcctgtcgatgggcggcctgatcggccagtggctgggtatccatgcaggtgagcgcctgcacagcctgaccctgtgcaacacggccgccaagatcgccaatgacgaggtgtggaacacccgtatcgacacggtactcaaaggcggccagcaggccatggtcgacctgcgcgatgcctccatcgcccgctggttcaccccgggctttgcccaggcgcaggcggagcaggcccagcgtatctgccagatgctggcgcaaaccagcccgcaaggctacgcaggcaactgtgcagcggtacgtgacgctgattatcgtgagcaactgggccgcatccaggtgcctgcgctgatcgttgccggtacccaagacgtggttaccacccctgagcatggccgcttcatgcaggccggtatccaaggtgccgagtacgtcgacttcccggcggcgcacctgtccaatgtcgagattggcgaggccttcagccgccgcgtgctcgatttcctgctggctcactga

SEQ ID NO: 22 - protein, 263 aa, Artificial Sequence
  PcaD amino acid sequence
MAHLQLADGVLNYQIDGPDDAPVLVLSNSLGTDLGMWDTQIPLWSQHFRVLRYDTRGHGASLVTEGPYSIEQLGRDVLALLDGLDIQKAHFVGLSMGGLIGQWLGIHAGERLHSLTLCNTAAKIANDEVWNTRIDTVLKGGQQAMVDLRDASIARWFTPGFAQAQAEQAQRICQMLAQTSPQGYAGNCAAVRDADYREQLGRIQVPALIVAGTQDVVTTPEHGRFMQAGIQGAEYVDFPAAHLSNVEIGEAFSRRVLDFLLAH

SEQ ID NO: 23 - DNA, 1194 bp, Artificial Sequence
  tdTer nucleotide sequence
atgatcgttaaaccgatggtgcgcaataacatttgtctgaatgcacatccgcagggttgtaaaaaaggtgttgaagatcagatcgagtacaccaaaaaacgtattacagccgaagttaaagccggtgcaaaagcaccgaaaaatgttctggttctgggttgtagcaatggttatggtctggcaagccgtattaccgcagcatttggttatggcgcagcaaccattggtgttagctttgaaaaagcaggtagcgaaaccaaatatggcacccctggttggtataataacctggcatttgatgaagcagcaaaacgtgaaggtctgtatagcgttaccattgatggtgatgcatttagcgacgaaattaaagcgcaggttattgaagaggccaaaaaaaagggcatcaaattcgacctgattgtttatagcctggcaagtccggttcgtaccgatccggataccggcatcatgcataaaagcgttctgaaaccgtttggcaaaacctttaccggcaaaaccgttgatccgtttaccggtgaactgaaagaaattagcgcagaaccggcaaatgatgaagaagcagcagcaaccgttaaagttatgggtggtgaagattgggaacgttggattaaacagctgagcaaagaaggtctgctggaagaaggttgtattaccctggcatatagttatattggtccggaagcaacccaggcactgtatcgtaaaggcaccattggtaaagcaaaagaacatctggaagccaccgcacatcgtctgaataaagaaaatccgagcattcgtgcatttgtgagcgttaataaaggtctggttacccgtgcaagcgcagtgattccggttattccgctgtatctggccagcctgtttaaagtgatgaaagaaaaaggtaaccacgaaggttgcattgagcagattacccgtctgtatgcagaacgtctgtatcgcaaagatggcaccattccggtggatgaagaaaatcgtattcgtatcgatgattgggagcttgaagaagatgttcagaaagcagttagcgcactgatggaaaaagtgaccggtgaaaatgcagaaagcctgaccgatctggcaggttatcgtcatgattttctggcaagtaatggctttgatgtggaaggcattaactatgaagcagaagtggaacgttttgaccgcatctaa

SEQ ID NO: 24 - protein, 397 aa, Artificial Sequence
  TdTer amino acid sequence
MIVKPMVRNNICLNAHPQGCKKGVEDQIEYTKKRITAEVKAGAKAPKNVLVLGCSNGYGLASRITAAFGYGAATIGVSFEKAGSETKYGTPGWYNNLAFDEAAKREGLYSVTIDGDAFSDEIKAQVIEEAKKKGIKFDLIVYSLASPVRTDPDTGIMHKSVLKPFGKTFTGKTVDPFTGELKEISAEPANDEEAAATVKVMGGEDWERWIKQLSKEGLLEEGCITLAYSYIGPEATQALYRKGTIGKAKEHLEATAHRLNKENPSIRAFVSVNKGLVTRASAVIPVIPLYLASLFKVMKEKGNHEGCIEQITRLYAERLYRKDGTIPVDEENRIRIDDWELEEDVQKAVSALMEKVTGENAESLTDLAGYRHDFLASNGFDVEGINYEAEVERFDRI

SEQ ID NO: 25 - DNA, 696 bp, Artificial Sequence
  pcaI nucleotide sequence
atgatcaacaaaacctatgagagcattgcaagcgcagttgaaggtattaccgatggtagcaccattatggttggtggttttggcaccgcaggtatgccgagcgaactgattgatggtctgattgcaaccggtgcacgtgatctgaccattatttctaataatgccggtaatggtgaaattggtctggcagcactgctgatggcaggtagcgttcgtaaagttgtttgtagctttccgcgtcagagcgatagctatgtttttgatgaactgtatcgcgcaggcaaaattgaactggaagttgttccgcagggtaatctggcagaacgtattcgtgcagccggtagcggtattggtgcattttttagcccgaccggttatggcaccctgctggccgaaggtaaagaaacccgtgaaattgatggccgtatgtatgttctggaaatgccgctgcatgccgattttgcactgattaaagcacataaaggtgatcgttggggcaatctgacctatcgtaaagcagcacgcaattttggtccgattatggcaatggcagcaaaaaccgcaattgcacaggttgatcaggttgttgaactgggtgaactggacccggaacatattatcacaccgggtatttttgttcagcgtgttgttgcagttaccggtgcagcagcaagcagcattgccaaagcagtttaa

SEQ ID NO: 26 - protein, 231 aa, Artificial Sequence
  PcaI amino acid sequence
MINKTYESIASAVEGITDGSTIMVGGFGTAGMPSELIDGLIATGARDLTIISNNAGNGEIGLAALLMAGSVRKVVCSFPRQSDSYVFDELYRAGKIELEVVPQGNLAERIRAAGSGIGAFFSPTGYGTLLAEGKETREIDGRMYVLEMPLHADFALIKAHKGDRWGNLTYRKAARNFGPIMAMAAKTAIAQVDQVVELGELDPEHIITPGIFVQRVVAVTGAAASSIAKAV

SEQ ID NO: 27 - DNA, 642 bp, Artificial Sequence
  pcaJ nucleotide sequence
atgaccatcaccaaaaaactgagccgtaccgaaatggcacagcgtgttgcagcagatattcaagaaggtgcatacgttaatctgggtattggtgcaccgaccctggttgcaaattatctgggtgataaagaagtgtttctgcatagcgaaaatggtctgctgggtatgggtccgagtccggcaccgggtgaagaggatgatgatctgattaatgcaggtaaacagcatgttaccctgctgaccggtggtgcattttttcatcatgcagatagctttagcatgatgcgtggtggtcatctggatattgcagttctgggtgcatttcaggttagcgttaaaggtgatctggcaaattggcataccggtgcagaaggtagcattccggcagttggtggtgcaatggatctggccaccggtgcacgtcaggtttttgttatgatggatcatctgaccaaaaccggtgaaagcaaactggttccggaatgcacatatccgctgaccggcattgcatgtgttagccgtatttataccgatctggccgttctggaagttacaccggaaggtctgaaagttgttgaaatttgtgccgatatcgatttcgatgaactgcagaaactgagcggtgttccgctgatcaaataa

SEQ ID NO: 28 - protein, 213 aa, Artificial Sequence
  PcaJ amino acid sequence
MTITKKLSRTEMAQRVAADIQEGAYVNLGIGAPTLVANYLGDKEVFLHSENGLLGMGPSPAPGEEDDDLINAGKQHVTLLTGGAFFHHADSFSMMRGGHLDIAVLGAFQVSVKGDLANWHTGAEGSIPAVGGAMDLATGARQVFVMMDHLTKTGESKLVPECTYPLTGIACVSRIYTDLAVLEVTPEGLKVVEICADIDFDELQKLSGVPLIK

SEQ ID NO: 29 - DNA, 855 bp, Artificial Sequence
  paaH1 nucleotide sequence
atgagcattcgtaccgttggtattgttggtgcaggcaccatgggtaatggtattgcacaggcatgtgcagttgttggtctgaatgttgttatggtggatattagtgatgcagccgttcagaaaggtgttgcaaccgttgccagcagcctggatcgtctgatcaaaaaagaaaaactgaccgaagcagataaagcaagcgcactggcacgtattaaaggtagcaccagctatgatgatctgaaagcaaccgatattgttattgaagcagccaccgaaaactatgacctgaaagtgaaaatcctgaaacaaatcgatggtatcgtgggcgaaaatgttattattgcaagcaataccagcagcatcagcattaccaaactggcagcagttaccagccgtgcagatcgttttattggtatgcattttttcaatccggttccggttatggcactggttgaactgattcgtggcctgcagaccagcgataccacccatgcagcagttgaagcactgagcaaacagctgggtaaatatccgattaccgtgaaaaattcaccgggttttgttgtgaatcgtattctgtgtccgatgatcaatgaagccttttgtgttctgggtgaaggtctggcaagtccggaagaaattgatgaaggtatgaaactgggttgcaatcatccgattggtccgctggcactggcagatatgattggtctggataccatgctggcagttatggaagttctgtataccgaatttgccgatccgaaatatcgtcctgccatgctgatgcgtgaaatggttgcagcaggttatctgggtcgtaaaaccggtcgtggtgtttatgtttatagcaaataa

SEQ ID NO: 30 - protein, 284 aa, Artificial Sequence
  PaaH1 amino acid sequence
MSIRTVGIVGAGTMGNGIAQACAVVGLNVVMVDISDAAVQKGVATVASSLDRLIKKEKLTEADKASALARIKGSTSYDDLKATDIVIEAATENYDLKVKILKQIDGIVGENVIIASNTSSISITKLAAVTSRADRFIGMHFFNPVPVMALVELIRGLQTSDTTHAAVEALSKQLGKYPITVKNSPGFVVNRILCPMINEAFCVLGEGLASPEEIDEGMKLGCNHPIGPLALADMIGLDTMLAVMEVLYTEFADPKYRPAMLMREMVAAGYLGRKTGRGVYVYSK

SEQ ID NO: 31 - DNA, 777 bp, Artificial Sequence
  ech nucleotide sequence (adipate pathway)
atgccgtatgaaaacattctggttgaaacccgtggtcgtgttggtctggttaccctgaatcgtccgaaagcactgaatgccctgaatgatgcactgatggatgaactgggtgcagcactgaccgcatttgatcaggatgaaggtattggtgcaattgttattaccggtagcgaacgtgcatttgcagccggtgcagatattggtatgatggcaaaatatagcttcatggacgtgtataaaggcgattatatcacccgtaattgggaaaccattcgcaaaattcgtaaaccggttattgccggtgttgcaggttatgcactgggtggtggttgtgaactggcaatgatgtgtgatattatcattgcagcagatagcgcgaaatttggtcagccggaagttaaactgggcaccatgcctggtgcgggtggcacccaacgtctgcctcgtgcagttagcaaagcaaaagcaatggatctgtgtctgaccagccgtatgatggatgcagcagaagcagaacgtagcggtctggtgagccgtgttgttccggcagataaactgctggatgaagttctggcagcagcagaaaccattgcaggttttagcctgccggttgttatgatgattaaagaaagcgttaatgcagcctatgaaaccaccctggcagaaggtgttcattttgaacgtcgtctgtttcatgcaacctttgcaagcgaagatcagaaagaaggtatggcagcatttgttgaaaaacgcagcccgaattttcagcaccgttaa

SEQ ID NO: 32 - protein, 258 aa, Artificial Sequence
  Ech amino acid sequence (adipate pathway)
MPYENILVETRGRVGLVTLNRPKALNALNDALMDELGAALTAFDQDEGIGAIVITGSERAFAAGADIGMMAKYSFMDVYKGDYITRNWETIRKIRKPVIAGVAGYALGGGCELAMMCDIIIAADSAKFGQPEVKLGTMPGAGGTQRLPRAVSKAKAMDLCLTSRMMDAAEAERSGLVSRVVPADKLLDEVLAAAETIAGFSLPVVMMIKESVNAAYETTLAEGVHFERRLFHATFASEDQKEGMAAFVEKRSPNFQHR

SEQ ID NO: 33 - DNA, 906 bp, Artificial Sequence
  ptb nucleotide sequence (adipate pathway)
atgatcaaaagcttcaacgagatcatcatgaaagtgaaaagcaaagaaatgaaaaaagttgccgttgcagttgcacaggatgaaccggtgctggaagcagttcgtgatgccaaaaaaaacggtattgcagatgcaattctggtgggtgatcatgatgaaattgttagcattgccctgaaaattggcatggatgtgaacgattttgaaatcgtgaatgagccgaatgtgaaaaaagcagcactgaaagcagttgaactggttagcaccggtaaagcagatatggttatgaaaggtctggttaataccgcaacctttctgcgtagcgttctgaataaagaagttggtctgcgtaccggtaaaaccatgagccatgttgcagtttttgaaaccgaaaaatttgatcgcctgctgtttctgaccgatgttgcatttaatacctatccggaactgaaagagaaaatcgatatcgttaacaacagcgtgaaagttgcacatgccattggtattgaaaatccgaaagtggcaccgatttgtgccgttgaagttattaatccgaaaatgccgagcaccctggatgcagcaatgctgagcaaaatgagcgatcgtggtcagattaaaggttgtgttgttgatggtccgctggcactggatattgcactgagcgaagaagcagcacatcataaaggtgttaccggtgaagttgcaggcaaagccgatatttttctgatgccgaatattgaaaccggcaacgtgatgtataaaaccctgacctataccaccgatagcaaaaatggtggtattctggttggcaccagcgcaccggttgttctgaccagccgtgcagatagccatgaaaccaaaatgaatagcattgcactggcagcactggttgcaggtaacaaataa

SEQ ID NO: 34 - protein, 301 aa, Artificial Sequence
  Ptb amino acid sequence (adipate pathway)
MIKSFNEIIMKVKSKEMKKVAVAVAQDEPVLEAVRDAKKNGIADAILVGDHDEIVSIALKIGMDVNDFEIVNEPNVKKAALKAVELVSTGKADMVMKGLVNTATFLRSVLNKEVGLRTGKTMSHVAVFETEKFDRLLFLTDVAFNTYPELKEKIDIVNNSVKVAHAIGIENPKVAPICAVEVINPKMPSTLDAAMLSKMSDRGQIKGCVVDGPLALDIALSEEAAHHKGVTGEVAGKADIFLMPNIETGNVMYKTLTYTTDSKNGGILVGTSAPVVLTSRADSHETKMNSIALAALVAGNK

SEQ ID NO: 35 - DNA, 1068 bp, Artificial Sequence
  buk1 nucleotide sequence (adipate pathway)
atgtatcgcctgctgattatcaatccgggtagcaccagcaccaaaattggtatttatgatgatgagaaagaaatctttgaaaaaaccctgcgtcatagcgcagaagaaatcgaaaaatacaacaccatcttcgaccagtttcagtttcgcaaaaatgttattctggatgccctgaaagaagccaacattgaagttagcagcctgaatgcagttgttggtcgtggtggtctgctgaaaccgattgttagcggcacctatgcagttaatcagaaaatgctggaagacctgaaagttggtgttcagggtcagcatgcaagcaatctgggtggtattattgcaaacgaaatcgccaaagaaattaacgtgcctgcctatattgttgatccggttgttgttgatgaactggatgaagtgagccgtattagcggtatggcagatattccgcgtaaaagcatttttcatgcgctgaatcagaaagcagttgcacgtcgttatgcaaaagaagtgggcaaaaaatacgaggatctgaatctgattgttgtgcacatgggtggtggcaccagcgttggcacccataaagatggtcgtgttattgaagtgaataataccctggatggtgaaggtccgtttagtccggaacgtagcggtggtgttccgattggtgatctggttcgtctgtgttttagcaacaaatatacctacgaagaagtgatgaaaaaaatcaacggtaaaggtggtgttgtgagctatctgaataccatcgattttaaagccgttgtggataaagcactggaaggtgacaaaaaatgtgccctgatttatgaagcctttacctttcaggttgcgaaagaaattggtaaatgtagcaccgttctgaaaggcaatgttgatgcaattattctgaccggtggtattgcctataatgaacacgtttgtaatgccattgaagatcgcgtgaaattcattgcaccggttgttcgttatggtggtgaagatgaactgctggcactggccgaaggtggcctgcgtgttctgcgtggcgaagaaaaagcaaaagaatacaaataa

SEQ ID NO: 36 - protein, 355 aa, Artificial Sequence
  Buk1 amino acid sequence (adipate pathway)
MYRLLIINPGSTSTKIGIYDDEKEIFEKTLRHSAEEIEKYNTIFDQFQFRKNVILDALKEANIEVSSLNAVVGRGGLLKPIVSGTYAVNQKMLEDLKVGVQGQHASNLGGIIANEIAKEINVPAYIVDPVVVDELDEVSRISGMADIPRKSIFHALNQKAVARRYAKEVGKKYEDLNLIVVHMGGGTSVGTHKDGRVIEVNNTLDGEGPFSPERSGGVPIGDLVRLCFSNKYTYEEVMKKINGKGGVVSYLNTIDFKAVVDKALEGDKKCALIYEAFTFQVAKEIGKCSTVLKGNVDAIILTGGIAYNEHVCNAIEDRVKFIAPVVRYGGEDELLALAEGGLRVLRGEEKAKEYK

SEQ ID NO: 37 - DNA, 738 bp, Artificial Sequence
  adc nucleotide sequence (adipate pathway)
atgttaaaggatgaagtaattaaacaaattagcacgccattaacttcgcctgcatttcctagaggaccctataaatttcataatcgtgagtattttaacattgtatatcgtacagatatggatgcacttcgtaaagttgtgccagagcctttagaaattgatgagcccttagtcaggtttgaaattatggcaatgcatgatacgagtggacttggttgttatacagaaagcggacaggctattcccgtaagctttaatggagttaagggagattatcttcatatgatgtatttagataatgagcctgcaattgcagtaggaagggaattaagtgcatatcctaaaaagctcgggtatccaaagctttttgtggattcagatactttagtaggaactttagactatggaaaacttagagttgcgacagctacaatggggtacaaacataaagccttagatgctaatgaagcaaaggatcaaatttgtcgccctaattatatgttgaaaataatacccaattatgatggaagccctagaatatgtgagcttataaatgcgaaaatcacagatgttaccgtacatgaagcttggacaggaccaactcgactgcagttatttgatcacgctatggcgccacttaatgatttgccagtaaaagagattgtttctagctctcacattcttgcagatataatattgcctagagctgaagttatatatgattatcttaagtaataa

SEQ ID NO: 38 - protein, 244 aa, Artificial Sequence
  Adc amino acid sequence (adipate pathway)
MLKDEVIKQISTPLTSPAFPRGPYKFHNREYFNIVYRTDMDALRKVVPEPLEIDEPLVRFEIMAMHDTSGLGCYTESGQAIPVSFNGVKGDYLHMMYLDNEPAIAVGRELSAYPKKLGYPKLFVDSDTLVGTLDYGKLRVATATMGYKHKALDANEAKDQICRPNYMLKIIPNYDGSPRICELINAKITDVTVHEAWTGPTRLQLFDHAMAPLNDLPVKEIVSSSHILADIILPRAEVIYDYLK

SEQ ID NO: 39 - DNA, 471 bp, Artificial Sequence
  HA controller (UniProt ID PP3359) nucleotide sequence
atggctaggtctgcccgtagtaccgacgacgcttgcgtggctgctcctgtgggagaaggggtgcttgaagacttgatcggctacgccttgcgacgcgcgcaattgaagctgtttcagaaccttattgcccggctctcggcccatgacctgcgcccggcccaattttccgccctggcgatcatcgaccagaaccccgggctgatgcaggccgacctggcgcgtgcgttggcaatcgaccccccgcaagtcgtgccaatgctgaacaaactggaagagcgcgcgctggccgtgcgcgtgcggtgcaaaccggacaagcgctcgtatgggattttcctgagcaaatcgggcgaggccctgttgaaggagttgaagcacatcgccgccgacagcgatcaccaggcgacatccaacctctcggatgacgaaaggactgaactgttgaggttattgaagaaaatctaccgggactga

SEQ ID NO: 40 - protein, 156 aa, Artificial Sequence
  HA controller (UniProt ID PP3359) amino acid sequence
MARSARSTDDACVAAPVGEGVLEDLIGYALRRAQLKLFQNLIARLSAHDLRPAQFSALAIIDQNPGLMQADLARALAIDPPQVVPMLNKLEERALAVRVRCKPDKRSYGIFLSKSGEALLKELKHIAADSDHQATSNLSDDERTELLRLLKKIYRD

SEQ ID NO: 41 - DNA, 6175 bp, Artificial Sequence
  pBAD Controller
ttatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggattctacctgacgctttttatcgcaactctctactgtttctccatacccgtttttttgggaattcaaaagatctaaagaggagaaaggatctatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaaggatctctgttctctaatgttaactccccctaacctgttgctttagttattcatttcctgtctcactttgccttaataccctacgttaaatgttactaatttgttgcttttgatcacaataagaaaacaatatgtcgcttttgtgcgcatttttcagaaatgtagatatttttagattatggctacgaaatgagcatcgccatgtcaccctacatctcataagggatcttttaagaaggagatatacatatgaataacgaagcccgctcagggtcgaccgaccctggccaacgtccgcgctaccgccaggtggccatcgggcatccccaggtgcaggtcagtcacgtcgacgacgtgctgcgcatgcaacctgtcgagccactggcgccgctgccggcgcgcctgctcgagcgcctggtgcattgggcccaggtgcgcccggacaccactttcatcgcggcacgccaggcagacggtgcctggcgttcgatcagctacgtgcagatgctcgccgatgtgcgcaccatcgccgccaacttgctaggactgggcctcagtgccgagcgcccgctggcgctgctttccggcaacgacatcgaacacctgcaaatcgccctcggcgccatgtatgccggtattgcctattgcccggtgtcgccggcctacgcgctgttgtcgcaagacttcgccaagttgcgccatgtctgcgaggtgctcacccccggagtggtcttcgtcagcgacagccagccgttccagcgcgccttcgaggcggtgctggacgattcggtcggcgtgatcagcgtgcgtggccaggtcgcaggtcgcccccatataagcttcgacagcctgttgcaaccgggtgacctggcggcggccgatgcggctttcgccgccaccgggccggacaccatcgccaaattcctcttcacctcgggctcgaccaagctgcccaaggcggtgatcaccacccagcgcatgctgtgcgccaatcagcagatgcttctgcagacttttccgacgttcgccgaggagccgccggtgctggtggactggctgccgtggaaccacacgttcggcggtagccacaacctcggcatcgtgctttacaacgggggcagtttctacctggacgccggcaagccgaccccgcaaggcttcgccgagaccttgcgcaatttgcgcgagatttcccccacggcctacctcaccgtacccaagggctgggaggaactggtcaaggcactggagcaggaccccgcgctacgcgaggtgttctttgcccgcatcaagctgttcttctttgccgccgcaggcctgtcgcaaagcgtctgggaccggctggaccgcattgccgagcaacactgtggcgaacgcatccgcatgatggccggccttggcatgaccgaagcctcgccatcgtgcaccttcaccaccgggcctttgtcgatggccggctatgtcgggctgccggcacctggctgcgaagtgaagctggtgccggtgggcgacaagctcgaggcgcgcttccgtggcccgcatatcatgccgggctactggcgctcgccgcagcagaccgccgaggcgttcgacgaggagggcttctactgttcgggcgacgcgttgaagctggccgatgccaggcagcccgagcttggcctgatgttcgatggccgtatcgctgaggacttcaaactttcgtccggggtattcgtcagtgtcgggccgctgcgcaaccgcgcagtgctggagggctcgccttacgtacaggacatcgtggtcaccgcgccggaccgtgaatgcctgggcctgctggtgttcccgcgtctgcccgagtgtcggcgcctggccgggctggcagaggatgccagcgatgcgcgggtgctggccaacgacaccgtgcgcagttggttcgctgactggctggagcgcttgaaccgcgatgcccaaggcaacgccagccgtatcgaatggctgtcgctgctggccgagccgccgtcgatcgacgccggtgaaatcaccgacaagggctcgatcaatcagcgcgccgtgctgcagcggcgcgccgctcaggtcgaggcgctgtaccgtggcgaagaccccgacgcattgcacgccaaggtgcggccttaaggatccaaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 42 - DNA, 5147 bp, Artificial Sequence
  HA Controller
ctcaggtttcatgctcctcgatcatgggtaataaagttacctattttgcctgtccttatgcgattcggctagagaggttctggaaaaaggcagcgcgcctaaccccaggacaaagataaaattgttaatggttaattgacataactaatttgacccgttagcgtggccccatcacctcgaacaacggatctaaagaggagaaaggatctatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaaggatctctgttctctaatgttaactccccctaacctgttgctttagttattcatttcctgtctcactttgccttaataccctacgttaaatgttactaatttgttgcttttgatcacaataagaaaacaatatgtcgcttttgtgcgcatttttcagaaatgtagatatttttagattatggctacgaaatgagcatcgccatgtcaccctacatctcataagggatcttttaagaaggagatatacatatgaataacgaagcccgctcagggtcgaccgaccctggccaacgtccgcgctaccgccaggtggccatcgggcatccccaggtgcaggtcagtcacgtcgacgacgtgctgcgcatgcaacctgtcgagccactggcgccgctgccggcgcgcctgctcgagcgcctggtgcattgggcccaggtgcgcccggacaccactttcatcgcggcacgccaggcagacggtgcctggcgttcgatcagctacgtgcagatgctcgccgatgtgcgcaccatcgccgccaacttgctaggactgggcctcagtgccgagcgcccgctggcgctgctttccggcaacgacatcgaacacctgcaaatcgccctcggcgccatgtatgccggtattgcctattgcccggtgtcgccggcctacgcgctgttgtcgcaagacttcgccaagttgcgccatgtctgcgaggtgctcacccccggagtggtcttcgtcagcgacagccagccgttccagcgcgccttcgaggcggtgctggacgattcggtcggcgtgatcagcgtgcgtggccaggtcgcaggtcgcccccatataagcttcgacagcctgttgcaaccgggtgacctggcggcggccgatgcggctttcgccgccaccgggccggacaccatcgccaaattcctcttcacctcgggctcgaccaagctgcccaaggcggtgatcaccacccagcgcatgctgtgcgccaatcagcagatgcttctgcagacttttccgacgttcgccgaggagccgccggtgctggtggactggctgccgtggaaccacacgttcggcggtagccacaacctcggcatcgtgctttacaacgggggcagtttctacctggacgccggcaagccgaccccgcaaggcttcgccgagaccttgcgcaatttgcgcgagatttcccccacggcctacctcaccgtacccaagggctgggaggaactggtcaaggcactggagcaggaccccgcgctacgcgaggtgttctttgcccgcatcaagctgttcttctttgccgccgcaggcctgtcgcaaagcgtctgggaccggctggaccgcattgccgagcaacactgtggcgaacgcatccgcatgatggccggccttggcatgaccgaagcctcgccatcgtgcaccttcaccaccgggcctttgtcgatggccggctatgtcgggctgccggcacctggctgcgaagtgaagctggtgccggtgggcgacaagctcgaggcgcgcttccgtggcccgcatatcatgccgggctactggcgctcgccgcagcagaccgccgaggcgttcgacgaggagggcttctactgttcgggcgacgcgttgaagctggccgatgccaggcagcccgagcttggcctgatgttcgatggccgtatcgctgaggacttcaaactttcgtccggggtattcgtcagtgtcgggccgctgcgcaaccgcgcagtgctggagggctcgccttacgtacaggacatcgtggtcaccgcgccggaccgtgaatgcctgggcctgctggtgttcccgcgtctgcccgagtgtcggcgcctggccgggctggcagaggatgccagcgatgcgcgggtgctggccaacgacaccgtgcgcagttggttcgctgactggctggagcgcttgaaccgcgatgcccaaggcaacgccagccgtatcgaatggctgtcgctgctggccgagccgccgtcgatcgacgccggtgaaatcaccgacaagggctcgatcaatcagcgcgccgtgctgcagcggcgcgccgctcaggtcgaggcgctgtaccgtggcgaagaccccgacgcattgcacgccaaggtgcggccttaaggatccaaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 43 - DNA, 2445 bp, Artificial Sequence
  fadE nucleotide sequence
atgatgattttgagtattctcgctacggttgtcctgctcggcgcgttgttctatcaccgcgtgagcttatttatcagcagtctgattttgctcgcctggacagccgccctcggcgttgctggtctgtggtcggcgtgggtactggtgcctctggccattatcctcgtgccatttaactttgcgcctatgcgtaagtcgatgatttccgcgccggtatttcgcggtttccgtaaggtgatgccgccgatgtcgcgcactgagaaagaagcgattgatgcgggcaccacctggtgggagggcgacttgttccagggcaagccggactggaaaaagctgcataactatccgcagccgcgcctgaccgccgaagagcaagcgtttctcgacggcccggtagaagaagcctgccggatggcgaatgatttccagatcacccatgagctggcggatctgccgccggagttgtgggcgtaccttaaagagcatcgtttcttcgcgatgatcatcaaaaaagagtacggcgggctggagttctcggcttatgcccagtctcgcgtgctgcaaaaactctccggcgtgagcgggatcctggcgattaccgtcggcgtgccaaactcattaggcccgggcgaactgttgcaacattacggcactgacgagcagaaagatcactatctgccgcgtctggcgcgtggtcaggagatcccctgctttgcactgaccagcccggaagcgggttccgatgcgggcgcgattccggacaccgggattgtctgcatgggcgaatggcagggccagcaggtgctggggatgcgtctgacctggaacaaacgctacattacgctggcaccgattgcgaccgtgcttgggctggcgtttaaactctccgacccggaaaaattactcggcggtgcagaagatttaggcattacctgtgcgctgatcccaaccaccacgccgggcgtggaaattggtcgtcgccacttcccgctgaacgtaccgttccagaacggaccgacgcgcggtaaagatgtcttcgtgccgatcgattacatcatcggcgggccgaaaatggccgggcaaggctggcggatgctggtggagtgcctctcggtaggccgcggcatcaccctgccttccaactcaaccggcggcgtgaaatcggtagcgctggcaaccggcgcgtatgctcacattcgccgtcagttcaaaatctctattggtaagatggaagggattgaagagccgctggcgcgtattgccggtaatgcctacgtgatggatgctgcggcatcgctgattacctacggcattatgctcggcgaaaaacctgccgtgctgtcggctatcgttaagtatcactgtacccaccgcgggcagcagtcgattattgatgcgatggatattaccggcggtaaaggcattatgctcgggcaaagcaacttcctggcgcgtgcttaccagggcgcaccgattgccatcaccgttgaaggggctaacattctgacccgcagcatgatgatcttcggacaaggagcgattcgttgccatccgtacgtgctggaagagatggaagcggcgaagaacaatgacgtcaacgcgttcgataaactgttgttcaaacatatcggtcacgtcggtagcaacaaagttcgcagcttctggctgggcctgacgcgcggtttaaccagcagcacgccaaccggcgatgccactaaacgctactatcagcacctgaaccgcctgagcgccaacctcgccctgctttctgatgtctcgatggcagtgctgggcggcagcctgaaacgtcgcgagcgcatctcggcccgtctgggggatattttaagccagctctacctcgcctctgccgtgctgaagcgttatgacgacgaaggccgtaatgaagccgacctgccgctggtgcactggggcgtacaagatgcgctgtatcaggctgaacaggcgatggatgatttactgcaaaacttcccgaaccgcgtggttgccgggctgctgaatgtggtgatcttcccgaccggacgtcattatctggcaccttctgacaagctggatcataaagtggcgaagattttacaagtgccgaacgccacccgttcccgcattggtcgcggtcagtacctgacgccgagcgagcataatccggttggcttgctggaagaggcgctggtggatgtgattgccgccgacccaattcatcagcggatctgtaaagagctgggtaaaaacctgccgtttacccgtctggatgaactggcgcacaacgcgctggtgaaggggctgattgataaagatgaagccgctattctggtgaaagctgaagaaagccgtctgcgcagtattaacgttgatgactttgatccggaagagctggcgacgaagccggtaaagttgccggagaaagtgcggaaagttgaagccgcgtaa

SEQ ID NO: 44 - protein, 814 aa, Artificial Sequence [sequence omitted]

SEQ ID NO: 45 - DNA, 1686 bp, Artificial Sequence
  fadD nucleotide sequence
ttgaagaaggtttggcttaaccgttatcccgcggacgttccgacggagatcaaccctgaccgttatcaatctctggtagatatgtttgagcagtcggtcgcgcgctacgccgatcaacctgcgtttgtgaatatgggggaggtaatgaccttccgcaagctggaagaacgcagtcgcgcgtttgccgcttatttgcaacaagggttggggctgaagaaaggcgatcgcgttgcgttgatgatgcctaatttattgcaatatccggtggcgctgtttggcattttgcgtgccgggatgatcgtcgtaaacgttaacccgttgtataccccgcgtgagcttgagcatcagcttaacgatagcggcgcatcggcgattgttatcgtgtctaactttgctcacacactggaaaaagtggttgataaaaccgccgttcagcacgtaattctgacccgtatgggcgatcagctatctacggcaaaaggcacggtagtcaatttcgttgttaaatacatcaagcgtttggtgccgaaataccatctgccagatgccatttcatttcgtagcgcactgcataacggctaccggatgcagtacgtcaaacccgaactggtgccggaagatttagcttttctgcaatacaccggcggcaccactggtgtggcgaaaggcgcgatgctgactcaccgcaatatgctggcgaacctggaacaggttaacgcgacctatggtccgctgttgcatccgggcaaagagctggtggtgacggcgctgccgctgtatcacatttttgccctgaccattaactgcctgctgtttatcgaactgggtgggcagaacctgcttatcactaacccgcgcgatattccagggttggtaaaagagttagcgaaatatccgtttaccgctatcacgggcgttaacaccttgttcaatgcgttgctgaacaataaagagttccagcagctggatttctccagtctgcatctttccgcaggcggtgggatgccagtgcagcaagtggtggcagagcgttgggtgaaactgaccggacagtatctgctggaaggctatggccttaccgagtgtgcgccgctggtcagcgttaacccatatgatattgattatcatagtggtagcatcggtttgccggtgccgtcgacggaagccaaactggtggatgatgatgataatgaagtaccaccaggtcaaccgggtgagctttgtgtcaaaggaccgcaggtgatgctgggttactggcagcgtcccgatgctaccgatgaaatcatcaaaaatggctggttacacaccggcgacatcgcggtaatggatgaagaaggattcctgcgcattgtcgatcgtaaaaaagacatgattctggtttccggttttaacgtctatcccaacgagattgaagatgtcgtcatgcagcatcctggcgtacaggaagtcgcggctgttggcgtaccttccggctccagtggtgaagcggtgaaaatcttcgtagtgaaaaaagatccatcgcttaccgaagagtcactggtgactttttgccgccgtcagctcacgggatacaaagtaccgaagctggtggagtttcgtgatgagttaccgaaatctaacgtcggaaaaattttgcgacgagaattacgtgacgaagcgcgcggcaaagtggacaataaagcctga

SEQ ID NO: 46 - protein, 561 aa, Artificial Sequence
  FadD amino acid sequence
MKKVWLNRYPADVPTEINPDRYQSLVDMFEQSVARYADQPAFVNMGEVMTFRKLEERSRAFAAYLQQGLGLKKGDRVALMMPNLLQYPVALFGILRAGMIVVNVNPLYTPRELEHQLNDSGASAIVIVSNFAHTLEKVVDKTAVQHVILTRMGDQLSTAKGTVVNFVVKYIKRLVPKYHLPDAISFRSALHNGYRMQYVKPELVPEDLAFLQYTGGTTGVAKGAMLTHRNMLANLEQVNATYGPLLHPGKELVVTALPLYHIFALTINCLLFIELGGQNLLITNPRDIPGLVKELAKYPFTAITGVNTLFNALLNNKEFQQLDFSSLHLSAGGGMPVQQVVAERWVKLTGQYLLEGYGLTECAPLVSVNPYDIDYHSGSIGLPVPSTEAKLVDDDDNEVPPGQPGELCVKGPQVMLGYWQRPDATDEIIKNGWLHTGDIAVMDEEGFLRIVDRKKDMILVSGFNVYPNEIEDVVMQHPGVQEVAAVGVPSGSSGEAVKIFVVKKDPSLTEESLVTFCRRQLTGYKVPKLVEFRDELPKSNVGKILRRELRDEARGKVDNKA

SEQ ID NO: 47 - DNA, 663 bp, Artificial Sequence
  atoD nucleotide sequence
atgaaaacaaaattgatgacattacaagacgccaccggcttctttcgtgacggcatgaccatcatggtgggcggatttatggggattggcactccatcccgcctggttgaagcattactggaatctggtgttcgcgacctgacattgatagccaatgataccgcgtttgttgataccggcatcggtccgctcatcgtcaatggtcgagtccgcaaagtgattgcttcacatatcggcaccaacccggaaacaggtcggcgcatgatatctggtgagatggacgtcgttctggtgccgcaaggtacgctaatcgagcaaattcgctgtggtggagctggacttggtggttttctcaccccaacgggtgtcggcaccgtcgtagaggaaggcaaacagacactgacactcgacggtaaaacctggctgctcgaacgcccactgcgcgccgacctggcgctaattcgcgctcatcgttgcgacacacttggcaacctgacctatcaacttagcgcccgcaactttaaccccctgatagcccttgcggctgatatcacgctggtagagccagatgaactggtcgaaaccggcgagctgcaacctgaccatattgtcacccctggtgccgttatcgaccacatcatcgtttcacaggagagcaaataa

SEQ ID NO: 48 - protein, 220 aa, Artificial Sequence
  AtoD amino acid sequence
MKTKLMTLQDATGFFRDGMTIMVGGFMGIGTPSRLVEALLESGVRDLTLIANDTAFVDTGIGPLIVNGRVRKVIASHIGTNPETGRRMISGEMDVVLVPQGTLIEQIRCGGAGLGGFLTPTGVGTVVEEGKQTLTLDGKTWLLERPLRADLALIRAHRCDTLGNLTYQLSARNFNPLIALAADITLVEPDELVETGELQPDHIVTPGAVIDHIIVSQESK

SEQ ID NO: 49 - DNA, 651 bp, Artificial Sequence
  atoA nucleotide sequence
atggatgcgaaacaacgtattgcgcgccgtgtggcgcaagagcttcgtgatggtgacatcgttaacttagggatcggtttacccacaatggtcgccaattatttaccggagggtattcatatcactctgcaatcggaaaacggcttcctcggtttaggcccggtcacgacagcgcatccagatctggtgaacgctggcgggcaaccgtgcggtgttttacccggtgcagccatgtttgatagcgccatgtcatttgcgctaatccgtggcggtcatattgatgcctgcgtgctcggcggtttgcaagtagacgaagaagcaaacctcgcgaactgggtagtgcctgggaaaatggtgcccggtatgggtggcgcgatggatctggtgaccgggtcgcgcaaagtgatcatcgccatggaacattgcgccaaagatggttcagcaaaaattttgcgccgctgcaccatgccactcactgcgcaacatgcggtgcatatgctggttactgaactggctgtctttcgttttattgacggcaaaatgtggctcaccgaaattgccgacgggtgtgatttagccaccgtgcgtgccaaaacagaagctcggtttgaagtcgccgccgatctgaatacgcaacggggtgatttatga

SEQ ID NO: 50 - protein, 216 aa, Artificial Sequence
  AtoA amino acid sequence
MDAKQRIARRVAQELRDGDIVNLGIGLPTMVANYLPEGIHITLQSENGFLGLGPVTTAHPDLVNAGGQPCGVLPGAAMFDSAMSFALIRGGHIDACVLGGLQVDEEANLANWVVPGKMVPGMGGAMDLVTGSRKVIIAMEHCAKDGSAKILRRCTMPLTAQHAVHMLVTELAVFRFIDGKMWLTEIADGCDLATVRAKTEARFEVAADLNTQRGDL

SEQ ID NO: 51 - DNA, 1206 bp, Artificial Sequence
  paaJ nucleotide sequence
atgcgtgaagcctttatttgtgacggaattcgtacgccaattggtcgctacggcggggcattatcaagtgttcgggctgatgatctggctgctatccctttgcgggaactgctggtgcgaaacccgcgtctcgatgcggagtgtatcgatgatgtgatcctcggctgtgctaatcaggcgggagaagataaccgtaacgtagcccggatggcgactttactggcggggctgccgcagagtgtttccggcacaaccattaaccgcttgtgtggttccgggctggacgcactggggtttgccgcacgggcgattaaagcgggcgatggcgatttgctgatcgccggtggcgtggagtcaatgtcacgggcaccgtttgttatgggcaaggcagccagtgcattttctcgtcaggctgagatgttcgataccactattggctggcgatttgtgaacccgctcatggctcagcaatttggaactgacagcatgccggaaacggcagagaatgtagctgaactgttaaaaatctcacgagaagatcaagatagttttgcgctacgcagtcagcaacgtacggcaaaagcgcaatcctcaggcattctggctgaggagattgttccggttgtgttgaaaaacaagaaaggtgttgtaacagaaatacaacatgatgagcatctgcgcccggaaacgacgctggaacagttacgtgggttaaaagcaccatttcgtgccaatggggtgattaccgcaggcaatgcttccggggtgaatgacggagccgctgcgttgattattgccagtgaacagatggcagcagcgcaaggactgacaccgcgggcgcgtatcgtagccatggcaaccgccggggtggaaccgcgcctgatggggcttggtccggtgcctgcaactcgccgggtgctggaacgcgcagggctgagtattcacgatatggacgtgattgaactgaacgaagcgttcgcggcccaggcgttgggtgtactacgcgaattggggctgcctgatgatgccccacatgttaaccccaacggaggcgctatcgccttaggccatccgttgggaatgagtggtgcccgcctggcactggctgccagccatgagctgcatcggcgtaacggtcgttacgcattgtgcaccatgtgcatcggtgtcggtcagggcatcgccatgattctggagcgtgtttga

SEQ ID NO: 52 - protein, 401 aa, Artificial Sequence
  PaaJ amino acid sequence
MREAFICDGIRTPIGRYGGALSSVRADDLAAIPLRELLVRNPRLDAECIDDVILGCANQAGEDNRNVARMATLLAGLPQSVSGTTINRLCGSGLDALGFAARAIKAGDGDLLIAGGVESMSRAPFVMGKAASAFSRQAEMFDTTIGWRFVNPLMAQQFGTDSMPETAENVAELLKISREDQDSFALRSQQRTAKAQSSGILAEEIVPVVLKNKKGVVTEIQHDEHLRPETTLEQLRGLKAPFRANGVITAGNASGVNDGAAALIIASEQMAAAQGLTPRARIVAMATAGVEPRLMGLGPVPATRRVLERAGLSIHDMDVIELNEAFAAQALGVLRELGLPDDAPHVNPNGGAIALGHPLGMSGARLALAASHELHRRNGRYALCTMCIGVGQGIAMILERV

SEQ ID NO: 53 - DNA, 1167 bp, Artificial Sequence
  sucC nucleotide sequence
atgaacttacatgaatatcaggcaaaacaactttttgcccgctatggcttaccagcaccggtgggttatgcctgtactactccgcgcgaagcagaagaagccgcttcaaaaatcggtgccggtccgtgggtagtgaaatgtcaggttcacgctggtggccgcggtaaagcgggcggtgtgaaagttgtaaacagcaaagaagacatccgtgcttttgcagaaaactggctgggcaagcgtctggtaacgtatcaaacagatgccaatggccaaccggttaaccagattctggttgaagcagcgaccgatatcgctaaagagctgtatctcggtgccgttgttgaccgtagttcccgtcgtgtggtctttatggcctccaccgaaggcggcgtggaaatcgaaaaagtggcggaagaaactccgcacctgatccataaagttgcgcttgatccgctgactggcccgatgccgtatcagggacgcgagctggcgttcaaactgggtctggaaggtaaactggttcagcagttcaccaaaatcttcatgggcctggcgaccattttcctggagcgcgacctggcgttgatcgaaatcaacccgctggtcatcaccaaacagggcgatctgatttgcctcgacggcaaactgggcgctgacggcaacgcactgttccgccagcctgatctgcgcgaaatgcgtgaccagtcgcaggaagatccgcgtgaagcacaggctgcacagtgggaactgaactacgttgcgctggacggtaacatcggttgtatggttaacggcgcaggtctggcgatgggtacgatggacatcgttaaactgcacggcggcgaaccggctaacttccttgacgttggcggcggcgcaaccaaagaacgtgtaaccgaagcgttcaaaatcatcctctctgacgacaaagtgaaagccgttctggttaacatcttcggcggtatcgttcgttgcgacctgatcgctgacggtatcatcggcgcggtagcagaagtgggtgttaacgtaccggtcgtggtacgtctggaaggtaacaacgccgaactcggcgcgaagaaactggctgacagcggcctgaatattattgcagcaaaaggtctgacggatgcagctcagcaggttgttgccgcagtggaggggaaataa

SEQ ID NO: 54 - protein, 388 aa, Artificial Sequence
  SucC amino acid sequence
MNLHEYQAKQLFARYGLPAPVGYACTTPREAEEAASKIGAGPWVVKCQVHAGGRGKAGGVKVVNSKEDIRAFAENWLGKRLVTYQTDANGQPVNQILVEAATDIAKELYLGAVVDRSSRRVVFMASTEGGVEIEKVAEETPHLIHKVALDPLTGPMPYQGRELAFKLGLEGKLVQQFTKIFMGLATIFLERDLALIEINPLVITKQGDLICLDGKLGADGNALFRQPDLREMRDQSQEDPREAQAAQWELNYVALDGNIGCMVNGAGLAMGTMDIVKLHGGEPANFLDVGGGATKERVTEAFKIILSDDKVKAVLVNIFGGIVRCDLIADGIIGAVAEVGVNVPVVVRLEGNNAELGAKKLADSGLNIIAAKGLTDAAQQVVAAVEGK

SEQ ID NO: 55 - DNA, 870 bp, Artificial Sequence
  sucD nucleotide sequence
atgtccattttaatcgataaaaacaccaaggttatctgccagggctttaccggtagccaggggactttccactcagaacaggccattgcatacggcactaaaatggttggcggcgtaaccccaggtaaaggcggcaccacccacctcggcctgccggtgttcaacaccgtgcgtgaagccgttgctgccactggcgctaccgcttctgttatctacgtaccagcaccgttctgcaaagactccattctggaagccatcgacgcaggcatcaaactgattatcaccatcactgaaggcatcccgacgctggatatgctgaccgtgaaagtgaagctggatgaagcaggcgttcgtatgatcggcccgaactgcccaggcgttatcactccgggtgaatgcaaaatcggtatccagcctggtcacattcacaaaccgggtaaagtgggtatcgtttcccgttccggtacactgacctatgaagcggttaaacagaccacggattacggtttcggtcagtcgacctgtgtcggtatcggcggtgacccgatcccgggctctaactttatcgacattctcgaaatgttcgaaaaagatccgcagaccgaagcgatcgtgatgatcggtgagatcggcggtagcgctgaagaagaagcagctgcgtacatcaaagagcacgttaccaagccagttgtgggttacatcgctggtgtgactgcgccgaaaggcaaacgtatgggccacgcgggtgccatcattgccggtgggaaagggactgcggatgagaaattcgctgctctggaagccgcaggcgtgaaaaccgttcgcagcctggcggatatcggtgaagcactgaaaactgttctgaaataa

SEQ ID NO: 56 - protein, 289 aa, Artificial Sequence
  SucD amino acid sequence
MSILIDKNTKVICQGFTGSQGTFHSEQAIAYGTKMVGGVTPGKGGTTHLGLPVFNTVREAVAATGATASVIYVPAPFCKDSILEAIDAGIKLIITITEGIPTLDMLTVKVKLDEAGVRMIGPNCPGVITPGECKIGIQPGHIHKPGKVGIVSRSGTLTYEAVKQTTDYGFGQSTCVGIGGDPIPGSNFIDILEMFEKDPQTEAIVMIGEIGGSAEEEAAAYIKEHVTKPVVGYIAGVTAPKGKRMGHAGAIIAGGKGTADEKFAALEAAGVKTVRSLADIGEALKTVLK

(19) United States
(12) Patent Application Publication
(10) Pub. No.: US 2023/0123501 A1
(43) Pub. Date: Apr. 20, 2023

(54) BIOSYNTHESIS OF COMMODITY CHEMICALS FROM OIL PALM EMPTY FRUIT BUNCH LIGNIN

(71) Applicant: National University of Singapore, Singapore (SG)

(72) Inventors: Tat Ming Samuel Lo, Singapore (SG); In Young Hwang, Singapore (SG); Matthew Wook Chang, Singapore (SG); Han-Saem Cho, Busan (KR)

(73) Assignee: National University of Singapore, Singapore (SG)

(21) Appl. No.: 17/905,565

(22) PCT Filed: Mar. 5, 2021
(86) PCT No.: PCT/SG2021/050114

§ 371 Date: Sep. 2, 2022

(30) Foreign Application Priority Data
Mar. 5, 2020 (SG) 10202002037R

(51) Int. Cl. C12P 7/04; C12N 9/00; C12N 9/90; C12N 9/88; C12N 9/02; C12N 15/52; C12N 15/70

(52) U.S. Cl. CPC C12P 7/04; C12N 9/93; C12Y 602/01034; C12N 9/90; C12Y 505/01002; C12N 9/88; C12Y 402/01017; C12Y 401/01044; C12N 9/0008; C12Y 102/01067; C12N 9/0071; C12Y 114/14; C12N 9/0069; C12Y 113/11003; C12N 15/52; C12N 15/70; C12N 2800/101

(57) ABSTRACT

The present invention relates to the metabolic engineering of a microbial host for the synthesis of value-added products from oil palm empty fruit brunches (OPEFBs). In one embodiment, the genetically engineered microorganism is Escherichia coli comprising a metabolic pathway consisting of 9 enzymes (11 genes) to utilize depolymerized lignin, namely vanillin, p-coumaric acid, p-hydroxybenzaldehyde, vanillic acid, p-hydroxybenzoic acid and ferulic acid, to produce β-ketoadipic acid, which can be subsequently converted into commercially important derivatives such as adipic acid and levulinic acid. The enzymes are feruloyl-CoA synthetase (fcs), enoyl-CoA hydratase (ech), vanillin dehydrogenase (vdh), vanillate O-demethylase (vanA; vanA and vanB), p-hydroxy benzoate hydroxylase (pobA), protocatechuate 3,4-dioxygenase {pcaGH; pcaG and pcaH), 3-carboxy-cis, cis-muconate cycloisomerase (pcaB), 4-carboxymuconolactone decarboxylase (pcaC), and β-ketoadipate enol-lactone hydrolase (pcaD).

### FIELD OF THE INVENTION

The present invention relates to methods of metabolic engineering a microbial host to synthesize chemicals from oil palm lignin. In particular, the invention relates to the production of adipic acid and levulinic acid from lignocellulose biomass in engineered Escherichia coli, and also provides recombinant cells made using such methods.

### BACKGROUND OF THE INVENTION

Lignocellulosic biomass is the most abundant renewable resource [Vardon et al., Energy & Environmental Science 8: 617-628 (2015); Deng et al., Biochemical Engineering Journal 105: 16-26 (2016)]. In particular, oil palm empty fruit bunches (OPEFBs), by-products products of palm oil production, are abundant lignocellulosic biomass largely burned for energy and regarded as waste. It is estimated that 1.1 tons of OPEFBs are generated per ton of oil palm produced, which amounts to 57 million tons annually [Murphy, Journal of Oil Palm Research 26: 1-24 (2014); Coral Medina et al., Bioresource Technology 194: 172-178 (2015)]. Given their availability in large quantities, OPEFBs are an attractive renewable lignocellulosic source that can serve as feedstock in biorefineries to produce value-added products. OPEFB can be converted to fermentable sugars [Li et al., Biotechnology and Applied Biochemistry 61: 426-431 (2014)] and lignin extract [Mohamad Ibrahim et al., CLEAN—Soil, Air, Water 36: 287-291 (2008)] through simple, cost-effective pre-treatments using chemicals and heat.

The use of OPEFB-derived fermentable sugars to support cell growth was explored in 2014 by Li et al., [Li et al., Biotechnology and Applied Biochemistry 61: 426-431 (2014)] who combined the use of dilute acid and whole fungal cell culture-catalyzed hydrolysis to extract fermentable sugars from OPEFBs. Hemicellulose was first stripped off from OPEFBs using acid hydrolysis, and the remaining cellulose-lignin complex was converted to glucose by the cellulases in the whole fungal cell culture. The OPEFB-derived sugars were subsequently used as a carbon source to cultivate Escherichia coli (E. coli) as a proof of concept. The use of OPEFB-derived lignin was explored in 2008 by Mohamad Ibrahim et al., where lignin was extracted from OPEFBs using 20% sulfuric acid, followed by nitrobenzene oxidation to break down the lignin. This extraction process releases a plethora of depolymerized lignin compounds, notably vanillin, p-coumaric acid, p-hydroxybenzaldehyde, vanillic acid, p-hydroxybenzoic acid and ferulic acid [Xu et al., ChemSusChem 5: 667-675 (2012)]. Vanillin and p-coumaric acid were the predominant degradation products, with concentrations of ˜1800 ppm (1.8 g/L) and ˜1000 ppm (1.0 g/L), respectively, [Mohamad Ibrahim et al., CLEAN—Soil, Air, Water 36: 287-291 (2008)]. These compounds are useful substrates for the production of commercially important organic acids. Taken together, the reported studies suggest that OPEFB derivatives can be potentially exploited for biorefinery processes, while microbial cells can be engineered to convert aromatic compounds into commodity chemicals while utilizing fermentable sugars for cell growth (FIG. 1). However, the effective utilization of depolymerized lignin is hampered by the need for a fractionation process for further valorization as value-added chemicals. There are tremendous efforts currently devoted to the development of efficient fractionation processes, although these costly processes can potentially undermine the economic feasibility and utility of OPEFB lignin.

Adipic acid is a high-demand commodity chemical that is used as a lubricant and as a precursor for nylon 6,6, polyester polyols and plasticizers [Vardon et al., Energy & Envronmental Science 8: 617-628 (2015)]. It has a market volume of 2.6 million tons per year [Polen et al., J Biotechnol 167: 75-84 (2013)] and was valued at USD 5.56 billion in 2016 [Research, Adipic Acid Market Size, Share & Trends Analysis Report by Application (Nylon 66 Fiber, Nylon 66 Resin, Polyurethane, Adipate Ester), By Region (APAC, North America, Europe, MEA, CSA), and Segment Forecasts, 2018-2024 (2018), worldwidewebdotgrandviewresearchdotcom/industry-analysis/adipic-acid-market]. On the other hand, levulinic acid had a market value of USD 164 million in 2020 [MarketWatch, Levulinic Acid Market Size 2020: Top Countries Data, Definition, Detailed Analysis of Current Industry Figures with Forecasts Growth By 2026 (2020), worldwidewebdotmarketwatchdotcom/press-release/levulinic-acid-market-size-2020-top-countries-data-definition-detailed-analysis-of-current-industry-figures-with-forecasts-growth-by-2026-2020-07-13] and is a versatile chemical having roles in industrial products such as resins, plasticizers, textiles, animal feed, coatings, and as an antifreeze [Ghorpade and Hanna, Cereals: Novel Uses and Processes, eds. G. M. Campbell, C. Webb & S. L. Mckee. (Boston, Mass.: Springer 433 US), 49-55 (1997)].

There is a need for improved economics of exploiting OPEFB and its under-utilized lignin fraction to produce commodity chemicals.

### SUMMARY OF THE INVENTION

Herein, a microbial-based bioprocess was devised that directly utilized unfractionated depolymerized OPEFB lignin as a substrate for commodity chemical production (FIG. 1). Towards this goal, the inventors engineered E. coli to have repurposed synthetic metabolic pathways, to serve as a multi-substrate bio-catalytic platform that can act on a variety of depolymerized OPEFB lignin constituents and funnel them towards the formation of a desired primary product.

Herein, the inventors have identified and constructed a metabolic pathway consisting of 9 enzymes (11 genes) that will enable E. coli to utilize all 6 constituents of depolymerized lignin to produce a versatile precursor molecule, β-ketoadipate, via the β-ketoadipic acid pathway [Wells and Ragauskas, Trends Biotechnol 30: 627-637 (2012)], and subsequently convert this intermediate into various commercially important derivatives such as adipic acid (reduction) and levulinic acid (decarboxylation).

To further improve the bioconversion and simplify the bioprocess, E. coli cells were engineered to have a regulatory element that functions as a dynamic sensor-based genetic controller. In engineered cells, the expression of enzymatic pathway genes is commonly controlled by an induction system, where an artificial inducer is used to activate enzyme expression. The use of such inducers, although effective, is less favorable due to their high cost and toxicity and the corresponding increased bioprocess complexity. To this end, a two-layer genetic controller [Lo et al., Cell Syst 3: 133-143 (2016)] was employed that regulated the enzyme expression and hence bioconversion based on the availability of nutrient and OPEFB lignin derivatives. This enabled the engineered E. coli to autonomously activate the bioconversion process when the substrates are available without the need for additional inducer.

Biosynthesis of commodity chemicals using this intermediate was also demonstrated, where up to 9.5 mg/L adipic acid and 455.57 mg/L levulinic acid were produced from the reconstituted OPEFB lignin cocktail under fermenter-controlled conditions.

The microbial host, E. coli MG 1655 was also subjected to strain optimization, where native E. coli genes that are involved in competing metabolic pathways were systematically deleted to improve bioproduction yield. Deletion of sucCD and atoDA resulted in the greatest improvement for adipic acid and levulinic acid production, respectively.

The disclosure relates to a platform using E. coli for bioproduction of commodity chemicals from OPEFB lignin.

Broadly, the platform may use E. coli MG 1655 and comprise;
(a) a complete heterologous metabolic pathway that utilizes the depolymerized lignin (vanillin, p-coumaric acid, p-hydroxybenzaldehye, vanillic acid, p-hydroxybenzoic acid and ferulic acid) to produce an intermediate precursor, β-ketoadipate and/or
(b) a pathway to convert β-ketoadipate to either adipic acid or levulinic acid and/or
(c) a genetic controller that regulates the expression of the heterologous genes when the substrates are present (i.e. hydroxycinnamic acids such as ferulic acid and p-coumaric acid) and/or
(d) sucCD or atoDA genes have been deleted to eliminate metabolic competition against bioproduction pathways.

According to a first aspect, the present invention provides an isolated genetically engineered microorganism for producing β-ketoadipic acid from depolymerized lignin, wherein the microorganism has been transformed by at least one polynucleotide molecule; the at least one polynucleotide molecule comprising heterologous β-ketoadipic acid pathway genes, feruloyl-CoA synthetase (fcs), enoyl-CoA hydratase (ech), vanillin dehydrogenase (vdh), vanillate O-demethylase (vanAB; vanA and vanB), p-hydroxybenzoate hydroxylase (pobA), protocatechuate 3,4-dioxygenase (pcaGH; pcaG and pcaH), 3-carboxy-cis,cis-muconate cycloisomerase (pcaB), 4-carboxymuconolactone decarboxylase (pcaC) and p-ketoadipate enol-lactone hydrolase (pcaD) operably linked to at least one promoter, wherein said genetically engineered microorganism can convert depolymerized lignin to β-ketoadipic acid.

In some embodiments, the isolated genetically engineered microorganism further comprises:
(a) heterologous β-ketoadipic acid utilization genes, β-ketoadipic acid succinyl-CoA transferase (pcaIJ; pcaI and pcaJ), 3-hydroxyacyl-CoA dehydrogenase (paaH1), enoyl-CoA hydratase (ech), trans-enoyl-CoA reductase (ter), phosphate butyryltransferase (ptb) and butyrate kinase 1 (buk1) operably linked to at least one promoter, wherein said genetically engineered microorganism can convert β-ketoadipic acid to adipic acid, and/or
(b) a heterologous β-ketoadipic acid utilization gene, acetoacetate decarboxylase (adc), operably linked to at least one promoter, wherein said genetically engineered microorganism can convert β-ketoadipic acid to levulinic acid.

In some embodiments:
the fcs gene encodes an amino acid sequence set forth in SEQ ID NO: 2; and/or
the ech gene encodes an amino acid sequence set forth in SEQ ID NO: 4; and/or
the vdh gene encodes an amino acid sequence set forth in SEQ ID NO: 6; and/or
the vanA gene encodes an amino acid sequence set forth in SEQ ID NO: 8; and/or
the vanB gene encodes an amino acid sequence set forth in SEQ ID NO: 10; and/or
the pobA gene encodes an amino acid sequence set forth in SEQ ID NO: 12; and/or
the pcaH gene encodes an amino acid sequence set forth in SEQ ID NO: 14; and/or
the pcaG gene encodes an amino acid sequence set forth in SEQ ID NO: 16; and/or
the pcaB gene encodes an amino acid sequence set forth in SEQ ID NO: 18; and/or
the pcaC gene encodes an amino acid sequence set forth in SEQ ID NO: 20; and/or
the pcaD gene encodes an amino acid sequence set forth in SEQ ID NO: 22; and/or
the ter gene encodes an amino acid sequence set forth in SEQ ID NO: 24; and/or
the pcaI gene encodes an amino acid sequence set forth in SEQ ID NO: 26; and/or
the pcaJ gene encodes an amino acid sequence set forth in SEQ ID NO: 28; and/or
the paaH1 gene encodes an amino acid sequence set forth in SEQ ID NO: 30; and/or
the ech gene encodes an amino acid sequence set forth in SEQ ID NO: 32; and/or
the ptb gene encodes an amino acid sequence set forth in SEQ ID NO: 34; and/or
the buk1 gene encodes an amino acid sequence set forth in SEQ ID NO: 36; and/or
the adc gene encodes an amino acid sequence set forth in SEQ ID NO: 38.

An asterisk at the C-terminal end of the sequence denotes a stop or termination codon.

In some embodiments:
the fcs gene comprises a nucleic acid sequence at least 80%, at least 85%, at least 90%, at least 95% sequence identity or 100% sequence identity to the polynucleotide sequence set forth in SEQ ID NO: 1; and/or
the ech gene comprises a nucleic acid sequence at least 80%, at least 85%, at least 90%, at least 95% sequence identity or 100% sequence identity to the polynucleotide sequence set forth in SEQ ID NO: 3; and/or
the vdh gene comprises a nucleic acid sequence at least 80%, at least 85%, at least 90%, at least 95% sequence identity or 100% sequence identity to the polynucleotide sequence set forth in SEQ ID NO: 5; and/or
the vanA gene comprises a nucleic acid sequence at least 80%, at least 85%, at least 90%, at least 95% sequence identity or 100% sequence identity to the polynucleotide sequence set forth in SEQ ID NO: 7; and/or
the vanB gene comprises a nucleic acid sequence at least 80%, at least 85%, at least 90%, at least 95% sequence identity or 100% sequence identity to the polynucleotide sequence set forth in SEQ ID NO: 9; and/or
the pobA gene comprises a nucleic acid sequence at least 80%, at least 85%, at least 90%, at least 95% sequence identity or 100% sequence identity to the polynucleotide sequence set forth in SEQ ID NO: 11; and/or
the pcaH gene comprises a nucleic acid sequence at least 80%, at least 85%, at least 90%, at least 95% sequence identity or 100% sequence identity to the polynucleotide sequence set forth in SEQ ID NO: 13; and/or
the pcaG gene comprises a nucleic acid sequence at least 80%, at least 85%, at least 90%, at least 95% sequence identity or 100% sequence identity to the polynucleotide sequence set forth in SEQ ID NO: 15; and/or
the pcaB gene comprises a nucleic acid sequence at least 80%, at least 85%, at least 90%, at least 95% sequence identity or 100% sequence identity to the polynucleotide sequence set forth in SEQ ID NO: 17; and/or
the pcaC gene comprises a nucleic acid sequence at least 80%, at least 85%, at least 90%, at least 95% sequence identity or 100% sequence identity to the polynucleotide sequence set forth in SEQ ID NO: 19; and/or
the pcaD gene comprises a nucleic acid sequence at least 80%, at least 85%, at least 90%, at least 95% sequence identity or 100% sequence identity to the polynucleotide sequence set forth in SEQ ID NO: 21; and/or
the ter gene comprises a nucleic acid sequence at least 80%, at least 85%, at least 90%, at least 95% sequence identity or 100% sequence identity to the polynucleotide sequence set forth in SEQ ID NO: 23; and/or
the pcaI gene comprises a nucleic acid sequence at least 80%, at least 85%, at least 90%, at least 95% sequence identity or 100% sequence identity to the polynucleotide sequence set forth in SEQ ID NO: 25; and/or
the pcaJ gene comprises a nucleic acid sequence at least 80%, at least 85%, at least 90%, at least 95% sequence identity or 100% sequence identity to the polynucleotide sequence set forth in SEQ ID NO: 27; and/or
the paaH1 gene comprises a nucleic acid sequence at least 80%, at least 85%, at least 90%, at least 95% sequence identity or 100% sequence identity to the polynucleotide sequence set forth in SEQ ID NO: 29; and/or
the ech gene comprises a nucleic acid sequence at least 80%, at least 85%, at least 90%, at least 95% sequence identity or 100% sequence identity to the polynucleotide sequence set forth in SEQ ID NO: 31; and/or
the ptb gene comprises a nucleic acid sequence at least 80%, at least 85%, at least 90%, at least 95% sequence identity or 100% sequence identity to the polynucleotide sequence set forth in SEQ ID NO: 33; and/or
the buk1 gene comprises a nucleic acid sequence at least 80%, at least 85%, at least 90%, at least 95% sequence identity or 100% sequence identity to the polynucleotide sequence set forth in SEQ ID NO: 35; and/or
the adc gene comprises a nucleic acid sequence at least 80%, at least 85%, at least 90%, at least 95% sequence identity or 100% sequence identity to the polynucleotide sequence set forth in SEQ ID NO: 37.

It would be understood that specific pathway genes described herein may be substituted by related genes encoding enzymes with equivalent catalytic function. It would also be understood that the nucleic acid sequence of genes used in the pathways of the invention may be codon optimized for the particular engineered host cell.

In some embodiments, the at least one promoter is modulated by a heterologous genetic controller. In some embodiments the at least one promoter is a constitutive promoter, such as T7. It would be understood that there are other promoters which may be suitable for the instant invention.

In some embodiments, the heterologous genetic controller is pBAD or hydroxycinnamic acid (HA). In some embodiments the pBAD controller comprises the nucleotide sequence set forth in SEQ ID NO: 41. In some embodiments the HA controller comprises the nucleotide sequence set forth in SEQ ID NO: 42.

In some embodiments, the isolated genetically engineered microorganism according to any aspect of the invention, further comprises an inactivated endogenous succinyl CoA synthetase gene, such as sucCD and/or an inactivated β-ketoadipyl-CoA thiolase gene, such as paaJ. In some embodiments the sucCD gene encodes an amino acid sequence set forth in SEQ ID NO: 54 and SEQ ID NO: 56 (SucC and SucD, respectively). In some embodiments the sucCD gene comprises a nucleic acid sequence at least 80%, at least 85%, at least 90%, at least 95% sequence identity or 100% sequence identity to the polynucleotide sequence set forth in SEQ ID NO: 53 and SEQ ID NO: 55 (sucC and sucD, respectively). In some embodiments the paaJ gene encodes an amino acid sequence set forth in SEQ ID NO: 52. In some embodiments the paaJ gene comprises a nucleic acid sequence at least 80%, at least 85%, at least 90%, at least 95% sequence identity or 100% sequence identity to the polynucleotide sequence set forth in SEQ ID NO: 51.

In some embodiments, the isolated genetically engineered microorganism according to any aspect of the invention, further comprises an inactivated endogenous Acyl CoA:acetate/3-ketoacid CoA transferase gene, such as atoDA. In some embodiments the atoDA gene encodes an amino acid sequence set forth in SEQ ID NO: 48 and SEQ ID NO: 50 (AtoD and AtoA, respectively). In some embodiments the atoDA gene comprises a nucleic acid sequence at least 80%, at least 85%, at least 90%, at least 95% sequence identity or 100% sequence identity to the polynucleotide sequence set forth in SEQ ID NO: 47 and SEQ ID NO: 49 (atoD and atoA, respectively).

In some embodiments, the isolated genetically engineered microorganism comprises a bacteria or yeast, preferably a bacteria such as Escherichia coli. In some embodiments the bacteria is Escherichia coli MG1655.

In some embodiments, the depolymerized lignin is from fibrous oil palm empty fruit bunches.

According to another aspect, the present invention provides the use of the isolated genetically engineered microorganism according to any aspect of the invention for the production of adipic acid, or for the production of levulinic acid.

According to another aspect, the present invention provides a recombinant vector comprising heterologous β-ketoadipic acid pathway genes, fcs, ech, vdh, vanAB (vanA and vanB), pobA, pcaGH (pcaG and pcaH), pcaB, pcaC and pcaD operably linked to at least one promoter, and/or
heterologous β-ketoadipic acid utilization genes, pcaIJ (pcaI and pcaJ), paaH1, ech, ter, ptb and buk1 operably linked to at least one promoter, and/or
a heterologous β-ketoadipic acid utilization gene, adc, operably linked to at least one promoter.

In some embodiments:
the fcs gene encodes an amino acid sequence set forth in SEQ ID NO: 2; and/or
the ech gene encodes an amino acid sequence set forth in SEQ ID NO: 4; and/or
the vdh gene encodes an amino acid sequence set forth in SEQ ID NO: 6; and/or
the vanA gene encodes an amino acid sequence set forth in SEQ ID NO: 8; and/or
the vanB gene encodes an amino acid sequence set forth in SEQ ID NO: 10; and/or
the pobA gene encodes an amino acid sequence set forth in SEQ ID NO: 12; and/or
the pcaH gene encodes an amino acid sequence set forth in SEQ ID NO: 14; and/or
the pcaG gene encodes an amino acid sequence set forth in SEQ ID NO: 16; and/or
the pcaB gene encodes an amino acid sequence set forth in SEQ ID NO: 18; and/or
the pcaC gene encodes an amino acid sequence set forth in SEQ ID NO: 20; and/or
the pcaD gene encodes an amino acid sequence set forth in SEQ ID NO: 22; and/or
the ter gene encodes an amino acid sequence set forth in SEQ ID NO: 24; and/or
the pcaI gene encodes an amino acid sequence set forth in SEQ ID NO: 26; and/or
the pcaJ gene encodes an amino acid sequence set forth in SEQ ID NO: 28; and/or
the paaH1 gene encodes an amino acid sequence set forth in SEQ ID NO: 30; and/or
the ech gene encodes an amino acid sequence set forth in SEQ ID NO: 32; and/or
the ptb gene encodes an amino acid sequence set forth in SEQ ID NO: 34; and/or
the buk1 gene encodes an amino acid sequence set forth in SEQ ID NO: 36; and/or
the adc gene encodes an amino acid sequence set forth in SEQ ID NO: 38.

In some embodiments:
the fcs gene comprises a nucleic acid sequence at least 80%, at least 85%, at least 90%, at least 95% sequence identity or 100% sequence identity to the polynucleotide sequence set forth in SEQ ID NO: 1; and/or
the ech gene comprises a nucleic acid sequence at least 80%, at least 85%, at least 90%, at least 95% sequence identity or 100% sequence identity to the polynucleotide sequence set forth in SEQ ID NO: 3; and/or
the vdh gene comprises a nucleic acid sequence at least 80%, at least 85%, at least 90%, at least 95% sequence identity or 100% sequence identity to the polynucleotide sequence set forth in SEQ ID NO: 5; and/or
the vanA gene comprises a nucleic acid sequence at least 80%, at least 85%, at least 90%, at least 95% sequence identity or 100% sequence identity to the polynucleotide sequence set forth in SEQ ID NO: 7; and/or
the vanB gene comprises a nucleic acid sequence at least 80%, at least 85%, at least 90%, at least 95% sequence identity or 100% sequence identity to the polynucleotide sequence set forth in SEQ ID NO: 9; and/or
the pobA gene comprises a nucleic acid sequence at least 80%, at least 85%, at least 90%, at least 95% sequence identity or 100% sequence identity to the polynucleotide sequence set forth in SEQ ID NO: 11; and/or
the pcaH gene comprises a nucleic acid sequence at least 80%, at least 85%, at least 90%, at least 95% sequence identity or 100% sequence identity to the polynucleotide sequence set forth in SEQ ID NO: 13; and/or
the pcaG gene comprises a nucleic acid sequence at least 80%, at least 85%, at least 90%, at least 95% sequence identity or 100% sequence identity to the polynucleotide sequence set forth in SEQ ID NO: 15; and/or
the pcaB gene comprises a nucleic acid sequence at least 80%, at least 85%, at least 90%, at least 95% sequence identity or 100% sequence identity to the polynucleotide sequence set forth in SEQ ID NO: 17; and/or
the pcaC gene comprises a nucleic acid sequence at least 80%, at least 85%, at least 90%, at least 95% sequence identity or 100% sequence identity to the polynucleotide sequence set forth in SEQ ID NO: 19; and/or
the pcaD gene comprises a nucleic acid sequence at least 80%, at least 85%, at least 90%, at least 95% sequence identity or 100% sequence identity to the polynucleotide sequence set forth in SEQ ID NO: 21; and/or
the ter gene comprises a nucleic acid sequence at least 80%, at least 85%, at least 90%, at least 95% sequence identity or 100% sequence identity to the polynucleotide sequence set forth in SEQ ID NO: 23; and/or
the pcaI gene comprises a nucleic acid sequence at least 80%, at least 85%, at least 90%, at least 95% sequence identity or 100% sequence identity to the polynucleotide sequence set forth in SEQ ID NO: 25; and/or
the pcaJ gene comprises a nucleic acid sequence at least 80%, at least 85%, at least 90%, at least 95% sequence identity or 100% sequence identity to the polynucleotide sequence set forth in SEQ ID NO: 27; and/or
the paaH1 gene comprises a nucleic acid sequence at least 80%, at least 85%, at least 90%, at least 95% sequence identity or 100% sequence identity to the polynucleotide sequence set forth in SEQ ID NO: 29; and/or
the ech gene comprises a nucleic acid sequence at least 80%, at least 85%, at least 90%, at least 95% sequence identity or 100% sequence identity to the polynucleotide sequence set forth in SEQ ID NO: 31; and/or
the ptb gene comprises a nucleic acid sequence at least 80%, at least 85%, at least 90%, at least 95% sequence identity or 100% sequence identity to the polynucleotide sequence set forth in SEQ ID NO: 33; and/or
the buk1 gene comprises a nucleic acid sequence at least 80%, at least 85%, at least 90%, at least 95% sequence identity or 100% sequence identity to the polynucleotide sequence set forth in SEQ ID NO: 35; and/or
the adc gene comprises a nucleic acid sequence at least 80%, at least 85%, at least 90%, at least 95% sequence identity or 100% sequence identity to the polynucleotide sequence set forth in SEQ ID NO: 37.

According to another aspect, the present invention provides a kit comprising an isolated genetically engineered microorganism according to any aspect of the invention, or a recombinant vector of any aspect of the invention.

According to another aspect, the present invention provides a method of producing β-ketoadipic acid from depolymerized lignin, comprising the step of culturing a plurality of genetically engineered microorganisms of any aspect of the invention under conditions for production of said β-ketoadipic acid.

According to another aspect, the present invention provides a method of producing adipic acid from depolymerized lignin, comprising the step of culturing a plurality of genetically engineered microorganisms of any aspect of the invention under conditions for production of said adipic acid.

According to another aspect, the present invention provides a method of producing levulinic acid from depolymerized lignin, comprising the step of culturing a plurality of genetically engineered microorganisms of any aspect of the invention under conditions for production of said levulinic acid.

In some embodiments, the method of any aspect of the invention further comprises isolating said product produced by the genetically engineered microorganisms.

In some embodiments, the microorganism comprises a bacterium, such as Escherichia coli, preferably Escherichia coli MG1655.

In some embodiments of the production methods of the invention, the depolymerized lignin is from fibrous oil palm empty fruit bunches.

The platform E. coli strain enables direct utilization of depolymerized lignin cocktail without fractionating into individual constituents. Moreover, in some embodiments the use of a genetic controller allows autonomous induction of gene expression, which reduces the cost of artificial inducers such as IPTG that is commonly used. An advantage of the platform is that it is customizable, where other pathways that can convert the precursor, β-ketoadipate, can be easily implemented for a chemical of interest.

### BRIEF DESCRIPTION OF THE FIGURES

FIG. 1 shows an overview of commodity chemical production using oil palm empty fruit bunches (OPEFBs). The aim of this study is indicated by the dotted box. The components of depolymerized lignin were reported in Mohamad Ibrahim et al., CLEAN—Soil, Air, Water 36: 287-291 (2008); and cultivation of bio-catalytic cells was reported in Li et al., Biotechnology and Applied Biochemistry 61: 426-431 (2014).

FIGS. 2A-B show the production of protocatechuic acid and β-ketoadipic acid. (A) Synthetic metabolic pathways involved in the conversion of OPEFB lignin derivatives to converge to a single intermediate (protocatechuic acid) (1) and a linear precursor (p-ketoadipic acid) (2) that are needed for adipic acid and levulinic acid production. Pathway genes (in bold) and enzymes are fcs (feruloyl-CoA synthetase), ech (enoyl-CoA hydratase), vdh (vanillin dehydrogenase), vanAB (vanillate O-demethylase), pobA (p-hydroxybenzoate hydroxylase), pcaGH (protocatechuate 3,4-dioxygenase), pcaB (3-carboxy-cis,cis-muconate cycloisomerase), pcaC (4-carboxymuconolactone decarboxylase) and pcaD (β-ketoadipate enol-lactone hydrolase). (B) production of protocatechuate (normalized to theoretical yield) using the EFB lignin constituents as substrates. FA, Ferulic acid; Van, Vanillin; VA, Vanillic acid; P-Ca, p-Coumaric acid; P-HB, p-Hydroxybenzaldehyde; P-HA, p-Hydroxybenzoic acid.

FIG. 3 shows a schematic diagram of the genetic constructs used in this study. Constructs used for controllers are shown in a), Hydroxycinnamic acid controller; and b), L-arabinose controller with a plasmid backbone from pBbS8a. Constructs used for OPEFB utilization and linearization are shown in c), Protocatechuic acid production system; and d), B-ketoadipic acid production system with a plasmid backbone from pBbE8k. Constructs used for organic acid production are shown in e), Levulinic acid production system; and f), Adipic production system with a plasmid backbone from Pacyc.

FIGS. 4A-C show construction and validation of a novel metabolic pathway to produce adipic acid using β-ketoadipic acid in E. coli. (A) Biosynthesis pathway for adipic acid in E. coli, and expression of the pathway enzymes and Western blot showing the products from each cell extract. (B) In vitro enzyme assay for β-ketoadipic acid succinyl-CoA transferases (PcaI and PcaJ), where formation of β-ketoadipyl-CoA:Mg²⁺ was measured at 305 nm and normalized to control and shown in relative activity (a.u.). A: pACYCDuet; I: PcaI; J: PcaJ; De: Denatured at 85° C. for 1 hr before use. (C) Activity of trans-enoyl-CoA reductases (Ter) was characterized by measuring the final production level of adipic acid. Ctrl: Cell extracts from pACYCD and pBbE8K; egTer: egTer extract was used as Ter; tdTer: tdTer extract was used as Ter; De: Denatured at 85° C. for 1 hr before use; N.D.: Not detected.

FIGS. 5A-C show the commodity chemical production from β-ketoadipic acid. (A) Synthetic metabolic pathways to convert the linear precursor (β-ketoadipic acid) to levulinic acid (1. Decarboxylation) and adipic acid (2. Reduction). Native enzymes that can potentially compete with the pathways have been identified in and deleted from the working host strain E. coli MG1655. Pathway genes (in bold) and enzymes are pcaIJ β-ketoacetate CoA transferase), paaH1 (3-ketoadipoyl-CoA reductase), ech* (enoyl-CoA hydratase), ter (2,3-dehydroadipoyl-CpA reductase), ptb (phosphate butyryltransferase), buk1 (butyrate kinase 1) and adc (acetoacetate decarboxylase). ech* is a different enoyl-CoA hydratase from the one used in the protocatechuic acid pathway. (B) Deletion strains were evaluated for adipic acid production. The deleted genes are fadE (acyl-CoA dehydrogenase), fadD (long-chain fatty acid CoA ligase), paaJ (β-ketoadipyl-CoA thiolase), and sucCD (succinyl-CoA synthetase). (C) The E. coli ΔatoDA strain was selected as it ensures metabolic flux towards levulinic acid production.

FIG. 6 shows the alignment of (A) PcaI with AtoD with 60.4% sequence similarity across 235 residues and (B) PcaJ with AtoA amino acid sequences with 52.5% sequence similarity across 236 residues.

FIGS. 7A-C show the controllers for enzyme regulation. (A) Genetic circuit controller systems. The L-arabinose (arabinose-inducible) controller system was compared with the hydroxycinnamic acid (lignin substrate-inducible) controller system. The genetic controller turns on expression of T7 polymerase, which in turn controls the expression of enzymes required for bioconversion of depolymerized lignin. (B) Bioconversion of p-coumarate to adipic acid using E. coli ΔsucCD with a controller system regulating enzyme expression. (C) Bioconversion of p-coumarate to levulinic acid using E. coli ΔatoDA with a controller system regulating enzyme expression.

FIGS. 8A-C show (A) Overall strategies used to improve biosynthesis of adipic or levulinic acid by engineered microbes using depolymerized EFB lignin derivatives. In bioreactors, (B) the biosynthesis of adipic acid and levulinic acid and (C) utilization of depolymerized OPEFB lignin derivatives were quantified. The 6 aromatic compounds are converted to protocatechuic acid (PCA) as outlined in the converging pathways in FIG. 2. pCA: p-coumaric acid, pHB: p-hydroxybenzaldehyde, pHA: p-hydroxybenzoic acid, FA: Ferulic acid, Van: Vanillin, VA: Vanillic acid.

FIG. 9 shows that optimizing the OPEFB lignin feed through balancing cell growth (indicated by OD₆₀₀) and production titer of adipic acid or levulinic acid at the given time points.

FIG. 10 shows a plasmid map of S8a-controller-T7RNAP.

FIG. 11 shows a plasmid map of E8k-BKA v3 C10.

FIG. 12 shows a plasmid map of pACYC-adipate (tdTer).

FIG. 13 shows a plasmid map of pACYC-T7p-adc (without lacO).

FIG. 14 shows the engineered production pathways for adipic acid and levulinic acid from depolymerized OPEFB lignin according to the invention.

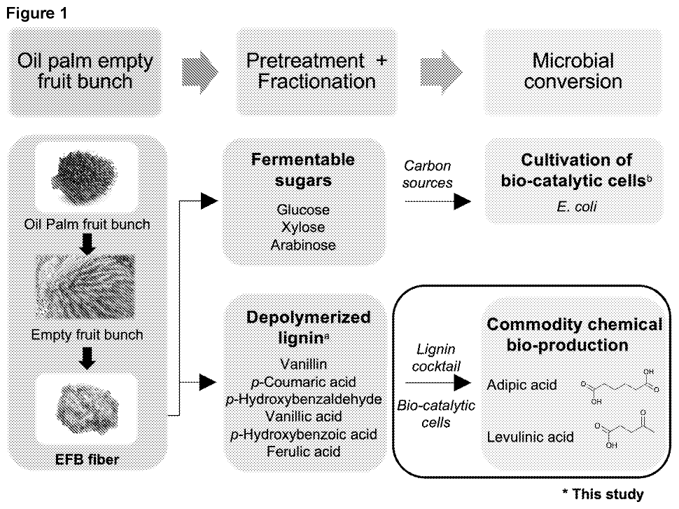

### DETAILED DESCRIPTION OF THE INVENTION

### Definitions

Certain terms employed in the specification, examples and appended claims are collected here for convenience.

It must be noted that as used herein and in the appended claims, the singular forms “a”, “an”, and “the” include plural reference unless the context clearly dictates otherwise.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

The term “isolated” is herein defined as a biological component (such as a nucleic acid, peptide or protein) that has been substantially separated, produced apart from, or purified away from other biological components in the cell of the organism in which the component naturally occurs, i.e., other chromosomal and extra-chromosomal DNA and RNA, and proteins. Nucleic acids, peptides and proteins which have been isolated thus include nucleic acids and proteins purified by standard purification methods. The term also embraces nucleic acids, peptides and proteins prepared by recombinant expression in a host cell as well as chemically synthesized nucleic acids.

The terms “nucleotide”, “nucleic acid” or “nucleic acid sequence”, as used herein, refer to an oligonucleotide, polynucleotide, or any fragment thereof, to DNA or RNA of genomic or synthetic origin which may be single-stranded or double-stranded and may represent the sense or the antisense strand, to peptide nucleic acid (PNA), or to any DNA-like or RNA-like material.

Several of the pathway enzymes comprise 2 subunits that are encoded by 2 separate genes. As used herein, the two subunit genes may be referred to as, for example, sucCD or alternatively separately as sucC and sucD. Similarly, the pathway enzymes may be referred to by their name, such as succinyl CoA synthetase or SucCD. In the present disclosure it would be understood that if an enzyme is to be inactivated, the inactivation may be achieved by various means including, for example, deletion of one or more genes encoding the enzyme subunits, or mutation of the gene coding sequence to produce inactive truncated or nonsense peptides.

As used herein, the term “operably linked” means that the components to which the term is applied are in a relationship that allows them to carry out their inherent functions under suitable conditions. For example, a control sequence which is “operably linked” to a protein coding sequence is ligated thereto, so that expression of the protein coding sequence is achieved under conditions compatible with the transcriptional activity of the control sequences. By way of an example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

The terms “amino acid” or “amino acid sequence,” as used herein, refer to an oligopeptide, peptide, polypeptide, or protein sequence, or a fragment of any of these, and to naturally occurring or synthetic molecules. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

As used herein, the terms “polypeptide”, “peptide” or “protein” refer to one or more chains of amino acids, wherein each chain comprises amino acids covalently linked by peptide bonds, and wherein said polypeptide or peptide can comprise a plurality of chains noncovalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. A “polypeptide”, “peptide” or “protein” can comprise one (termed “a monomer”) or a plurality (termed “a multimer”) of amino acid chains.

Bibliographic references mentioned in the present specification are for convenience listed in the form of a list of references and added at the end of the examples. The whole content of such bibliographic references is herein incorporated by reference. Any discussion about prior art is not an admission that the prior art is part of the common general knowledge in the field of the invention.

A vector can include one or more catalytic enzyme nucleic acid(s) in a form suitable for expression of the nucleic acid(s) in a host cell. Preferably the recombinant expression vector includes one or more regulatory sequences operatively linked to the nucleic acid sequence(s) to be expressed. The term “regulatory sequence” includes promoters, enhancers, ribosome binding sites and/or IRES elements, and other expression control elements (e.g., polyadenylation signals). Regulatory sequences include those which direct constitutive expression of a nucleotide sequence such as the T7 promoter disclosed in the Examples herein. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., catalytic enzyme proteins).

The recombinant expression vectors of the invention can be designed for expression of catalytic enzyme proteins in prokaryotic or eukaryotic cells, more particularly prokaryotic cells. For example, polypeptides of the invention can be expressed in bacteria (e.g., cyanobacteria) or yeast cells. Suitable host cells are discussed further in Goeddel, (1990) Gene Expression Technology: Methods in Enzymology 185, Academic Press, San Diego, Calif.

The methods described hereinbefore make use of enzymes to catalyse a sequence of reactions. While these reactions may be performed individually or, more particularly, two or more of them in combination, it is particularly preferred that all of the reactions are combined into a cascade reaction sequence that provides the product from the initial starting material in one pot, thereby eliminating the need for isolation of the intermediates and, potentially, increasing the overall yield of the reaction sequence.

The engineered cells of the invention further comprise inactivated genes to limit the utilization by the host cell of intermediate compounds in other biosynthetic pathways and reducing yield of the target final products. The engineered cells may comprise an inactivated endogenous succinyl CoA synthetase gene, such as sucCD (sucC, SEQ ID NO: 53 and sucD, SEQ ID NO: 55) and/or an inactivated β-ketoadipyl-CoA thiolase gene, such as paaJ, (SEQ ID NO: 51) if adipic acid is the intended product, or an inactivated endogenous Acyl CoA:acetate/3-ketoacid CoA transferase gene, such as atoDA (atoD, SEQ ID NO: 47 and atoA, SEQ ID NO: 49), if levulinic acid is the intended target product.

Having now generally described the invention, the same will be more readily understood through reference to the following examples which are provided by way of illustration, and are not intended to be limiting of the present invention.

A person skilled in the art will appreciate that the present invention may be practiced without undue experimentation according to the methods given herein. The methods, techniques and chemicals are as described in the references given or from protocols in standard biotechnology and molecular biology textbooks.

### EXAMPLES

### Example 1

### Materials and Methods

### Plasmid Assembly

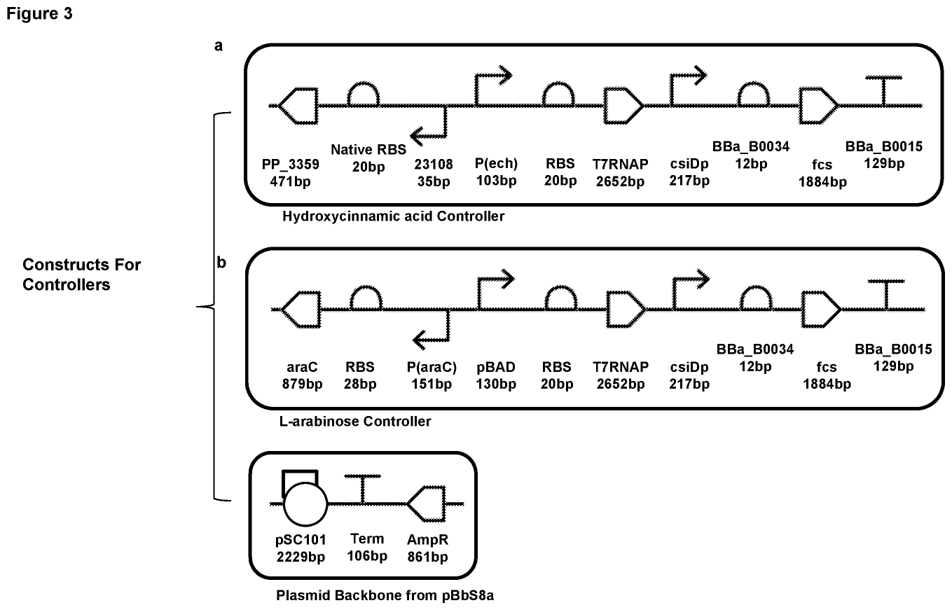

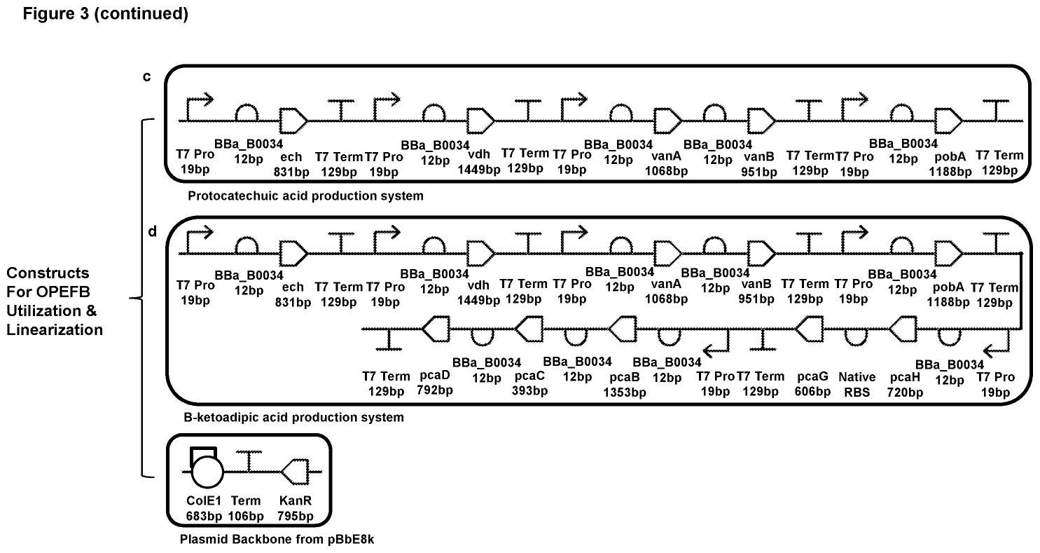

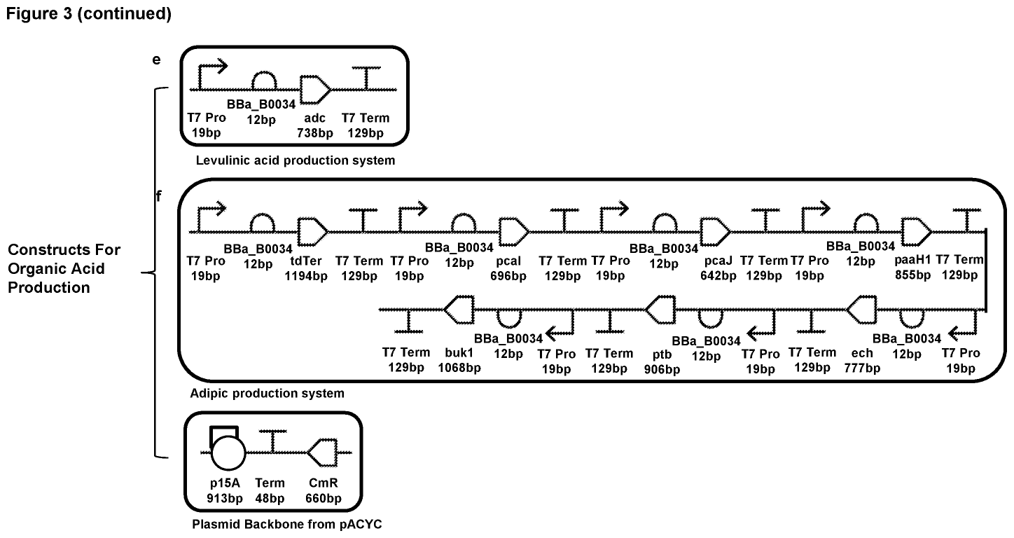

The plasmid backbones used in this study were the BgIBrick vectors [Lee et al., Journal of Biological Engineering 5: 12 (2011)] pBbE8k and pBbE8a from the Joint BioEnergy Institute, U.S.A. Cloning and modification of DNA parts such as promoters, genes, and terminators required the use of the splicing overlap extension (SOE) technique [Heckman and Pease, Nature Protocols 2: 924-932 (2007)]. Biological parts were either PCR-cloned from genomic templates of P. putida KT2440 and E. coli K-12 MG1655 or SOE-assembled using gene fragments (gBlocks) from Integrated DNA Technologies, U.S.A. They were converted into the BgIBrick standard, which was comprised of universal linkers such as EcoRI, BgIII, BamHI and Xhol restriction sites for assembly. The standard BgIBrick assembly method, described by Anderson et al. (2010) [Anderson et al., Journal of Biological Engineering 4: 1 (2010)], was used to assemble the genetic constructs listed in FIG. 3. Recombinant BgIBrick plasmids were chemically transformed into E. coli K-12 TOP10 (Invitrogen, U.S.A.). Transformed E. coli strains were first cultivated in Luria-Bertani (LB) broth at 37° C. and 225 rpm and screened via colony PCR. Gene deletion was introduced using a previously described method [Datsenko and Wanner, PNAS USA 97: 6640-6645 (2000)], incorporated herein by reference. List of bacterial strains and plasmids used in this study are listed in Table 1.

TABLE 1

| Strain/Plasmid | Description | Source |
|---|---|---|
| E. coli K-12 | Wild-type | CGSC |
| MG1655 | | 6300 |
| ΔfadE | MG1655, ΔfadE | This study |
| ΔfadED | MG1655, ΔfadEΔfadD | This study |
| ΔatoDA | MG1655, ΔatoDΔatoA | This study |
| ΔpaaJ | MG1655, ΔpaaJ | This study |
| ΔsucCD | MG1655, ΔsucCΔsucD | This study |
| HA controller | pSC101 plasmid backbone with; Amp^{R}, | 1 |
| | P_{J23108}-PP_3359, P_{ech}-T7RNAP, | |
| | P_{csiD}-fcs | |
| L-Ara | pSC101 plasmid backbone with; Amp^{R}, | 1 |
| controller | P_{araC}-araC, P_{BAD}-T7RNAP, P_{csiD}-fcs | |
| β-ketoadipic | pBbE8k plasmid backbone with; Kan^{R}, P_{T7}- | This study |
| acid | ech-vdh-vanA-vanB-pobA-pcaH-pcaG- | |
| | pcaB-pcaC-pcaD | |
| Adipic | pACYC plasmid backbone with; Cm^{R}, P_{T7}- | This study |
| acid | tdTer-pcaI-pcaJ-paaH1-ech*-ptb-buk1 | |

1 Lo, et al.. (2016). A Two-Layer Gene Circuit for Decoupling Cell Growth from Metabolite Production. Cell Syst 3: 133-143.

### Preparation of Cell Extracts for In Vitro Enzymatic Assays for Adipic Acid Pathway Validation

E. coli BL21(DE3) was transformed with each plasmid bearing one of the PcaI-, PcaJ-, PaaH-, Ech-, egTer-, tdTer-, Ptb-, or Buk1-encoding genes in either pBbE8k or pACYCDuet-1 (Novagen, Germany). Each gene was from P. putida KT2440 (PcaI and PcaJ), Ralstonia eutropha (PaaH1), R. eutropha H16 (Ech), Euglena gracilis (egTer), Treponema denticola (TdTer) or Clostridium acetobutylicum (Ptb and Buk1). The seed cultures for the transformants were prepared by overnight cultivation in LB medium supplemented with the appropriate antibiotic (30 μg/L kanamycin or 50 μg/L ampicillin) at 37° C. and 225 rpm. The seed cultures were diluted 1:100 (v/v) into Terrific Broth medium supplemented with appropriate antibiotics (30 μg/L kanamycin or 50 μg/L ampicillin) and cultivated at 37° C. and 225 rpm. The diluted E. coli cultures were induced with 0.1 mM IPTG at OD₆₀₀ 0.5-1.0 and cultivated at 16° C. and 225 rpm for 24 h. The cultures were harvested and resuspended with 0.5 mL of lysis buffer (20 mM Tris-HCl, 200 mM NaCl, 1 mM DTT, and 10% (v/v) glycerol, pH 7.5, final concentrations) and incubated at 25° C. and 150 rpm for 1 h with 1.5 mg/mL lysozyme. After the addition of 0.1% Triton X-100 and 1×protease inhibitor (Promega), the soluble fraction of crude cell extract was prepared by using FastPrep-24™ 5G (MP Biomedicals) and acid-washed beads (≤106 μm) (Sigma-Aldrich) at 6.5 m/s and 45 s, followed by centrifugation at 4° C. and 13,000 rpm for 10 min. Total proteins in the soluble extracts were manually quantified by using Bradford reagent (Sigma-Aldrich). The overexpression of each gene was verified by SDS-PAGE.

### In Vitro Enzyme Assay for β-Ketoadipic Acid Succinyl-CoA Transferases

The activities of β-ketoadipic acid succinyl-CoA transferase (subunit, PcaI and subunit, PcaJ) were determined as described previously [MacLean et al., Appl Environ Microbiol 72: 5403-5413 (2006)], incorporated herein by reference, with slight modifications. Briefly, the reaction was started with the addition of a reaction mixture (200 mM Tris-HCl, 0.4 mM succinyl-CoA, 40 mM MgSO₄, and 1 g/L β-ketoadipic acid, pH 8.0, final concentrations) to aliquots of cell extracts to a final volume of 0.1 mL. The formation of β-ketoadipyl-CoA:Mg²⁺ was monitored at 305 nm and 30° C. for 4 min by using a Biotek Synergy H1m microplate reader.

### In Vitro Adipate Production

In vitro adipate production was performed as described previously [Yu et al., Biotechnol Bioeng 111: 2580-2586 (2014)], incorporated herein by reference, with the following modifications. Each cell extract (equivalent to 0.05 mg of total protein) for PcaI, PcaJ, PaaH1, Ech, Ter (either egTer or tdTer), Ptb, and Buk1 was added into a reaction mixture (50 mM potassium phosphate buffer, 0.4 mM succinyl-CoA, 4 mM NADH, 2 mM ADP, and 0.5 g/L β-ketoadipic acid, pH 7.0, final concentrations) to a final volume of 0.2 mL and incubated for 24 h at room temperature. Subsequently, each sample was mixed with 0.2 mL of 1 M HCl and internal standard (1,14-tetradecanedioic acid) to a volume of 0.5 mL and vortexed thoroughly for 30 s. After the addition of 0.5 mL of ethyl acetate, the sample was vortexed thoroughly for 1 min, followed by centrifugation at 13,000 rpm for 1 min. Then, 0.35 mL of the ethyl acetate fraction was aliquoted and evaporated by using a rotary evaporator, followed by resuspension in 0.04 mL of ethyl acetate. The resuspended sample was mixed with N,O-bis(trimethylsilyl)trifluoroacetamide (BSTFA) at a 1:1 (v/v) ratio and derivatized at room temperature for 24 h. The formation of adipic acid was analyzed using GC-MS.

### Shake-Flask Adipic Acid Production for Engineered Host Screening

Overnight seed cultures were diluted 1:100 (v/v) into 50 mL of M9 medium supplemented with 0.2% (w/v) glucose, 0.2% (w/v) casamino acids and the appropriate antibiotics (100 μg/L carbenicillin, 50 μg/L kanamycin, and 25 μg/L chloramphenicol) in 250 mL baffled flasks and cultured at 30° C. and 225 rpm. The engineered E. coli cultures were induced with 0.2% (w/v) L-arabinose at OD₆₀₀ 1.2-1.5, which was followed by the addition of the p-coumaric acid substrate to a final concentration of 0.1% (w/v). Sampling was performed at 18 h and 36 h. The formation of adipic acid was analyzed by using GC-MS.

### Bioconversion by Engineered Cells Bearing Different Controllers

Engineered E. coli MG1655 cells were first grown in M9 medium (supplemented with 0.2% (w/v) glucose and 0.2% (w/v) casamino acids) to the exponential phase at OD₆₀₀ 1.0. The inoculums were added to shaken flasks (37° C., 225 rpm) to a final concentration of OD₆₀₀ 0.01, with each flask containing 50 mL of M9 medium supplemented with 0.2% (w/v) glucose as the carbon source, 0.2% (w/v) casamino acids and the relevant lignin derivatives as substrates. p-Coumaric acid (Sigma-Aldrich, U.S.A.) was first dissolved in dimethyl sulfoxide (DMSO) to a stock concentration of 10% (w/v) before being added to the M9 medium to a final concentration of 0.1% (w/v). The L-arabinose system and HA-controlled system were induced with 0.2% (w/v) L-arabinose or 0.1% (w/v) p-coumarate, respectively, after reaching OD₆₀₀ 1.0. One milliliter of the biotransformation culture was extracted at each time point (18 h and 36 h) for GC-MS measurements.

### Reconstitution of OPEFB Depolymerized Lignin Cocktail

OPEFB depolymerized lignin cocktail was reconstituted based on the identified concentrations of aromatic compounds reported in Mohamad Ibrahim et al., [Mohamad Ibrahim et al., CLEAN—Soil, Air, Water 2=36: 287-291 (2008)]. In brief, individual compounds of OPEFB were prepared separately and subsequently mixed together to give the final concentrations stated in Table 2.

TABLE 2
Production of protocatechuic acid (PCA) using EFB lignin
derivatives as substrates at 36 h. The concentration
of substrates used represents the concentration found
in the pre-treated, depolymerized EFB lignin.

| | Substrate | PCA | % |
|---|---|---|---|
| Substrates | used (mg) | (mg/L) | Conversion |
| Vanillin | 1800 | 49.6 ± 6.7 | 2.7 |
| p-coumaric acid | 1000 | 273.8 ± 41.7 | 29.1 |
| p-hydroxybenzaldehyde | 320 | 166.2 ± 28.9 | 41.1 |
| Vanillic acid | 110 | 112.5 ± 1.4 | 100.0 |
| p-hydroxybenzoic | 18 | 7.2 ± 6.0 | 36.0 |
| acid | | | |
| Ferulic acid | 13 | 7.2 ± 1.7 | 69.6 |
| EFB (All) | 3261 | 380.2 ± 10.2 | 11.5 |

All the individual compounds were purchased from Sigma-Aldrich with a purity of >97% and prepared in DMSO at a concentration that limits DMSO to 1% (v/v) in the final lignin cocktail solution. All the stock solutions of the compounds were kept at 4° C. in aliquots prior to use. Ten milliliter of lignin cocktail was prepared to comprise; 1.8 g of vanillin (Cat. No. 94752), 1 g of p-coumaric acid (≥98% (HPLC), Cat. No. C9008), 320 mg of p-hydroxybenzaldehyde (4-hydroxybenzoic acid; ≥99%, Cat. No. 240141), 110 mg of vanillic acid (4-hydroxy-3-methoxybenzoic acid; ≥97% (HPLC), Cat. No. 94770), 18 mg of p-hydroxybenzoic acid (3,4-dihydroxybenzoic acid; ≥98%, Cat. No. 37580) and 13 mg of ferulic acid (trans-ferulic acid; 99%, Cat. No. 128708) in DMSO. The solution was vortexed to ensure homogenized mixture. The cocktail was diluted 100-fold in the reaction volume to result in the final substrate concentration and referred as ‘1×OPEFB’.

### Bioconversion by Engineered Cells Using OPEFBs in Batch Culture

Batch fermentation was carried out in a 5 L working volume BIOSTAT® B-DCU II benchtop bioreactor (Sartorius Stedim) for bioconversion to generate adipic acid or levulinic acid. The temperature was maintained at 30° C., and the pH was controlled at 7.0 by automated addition of acid (1 M H₂SO₄) and base solutions (1 M NaOH). Oxygen was constantly supplied at 10 L/min, and the impeller speed was set to 400 rpm to ensure homogenous aeration. Antifoam agent (200 μL) was added to the culture to prevent excessive foaming. A seed culture of relevant engineered E. coli MG1655 cells was cultured at 30° C. overnight and subsequently transferred into 1 L of fresh culture media containing 3 antibiotics (100 mg/L carbenicillin, 50 mg/L kanamycin, and 25 mg/L chloramphenicol). For fermentation of engineered E. coli with an L-arabinose-controlled circuit, substrate (OPEFB lignin cocktail) and inducer (0.2% (w/v) L-arabinose) were added 4 h post-inoculation as the culture reached late log phase. For fermentation of engineered E. coli with an HA-controlled circuit, the substrate was fed into the vessel immediately following inoculation. Aliquots of samples were taken at 18 h, 36 h and 42 h for further analysis using GC-MS and absorbance measurements at 600 nm. Batch fermentations were run in duplicate, and the results are reported as the average with standard deviation.

### HPLC Quantification

Ferulic acid, p-coumaric acid, vanillin, vanillic acid, p-hydroxybenzaldehyde, p-hydroxybenzoic acid, and protocatechuic acid quantification were carried out using the protocol adopted by Barghini et al. [Barghini et al., Microbial Cell Factories 6: 13 (2007)], incorporated herein by reference, with modifications. First, 0.4 mL of the extracted batch culture was filter-sterilized with a 0.22 μm filter (Sartorius Stedim, Germany) before analysis by an Agilent 1260 HPLC apparatus equipped with an Inertsil ODS3 C18 reverse-phase column (length 250 mm, diameter 4.6 mm and particle size 5 μm) and a diode array detector (DAD). Compounds in the filtered culture were eluted with an isocratic pressure of 150 bars, a mobile phase comprising an aqueous solution of 35% methanol and 1% acetic acid, and a flow rate of 1 mL/min. Detection was performed at UV wavelengths of 300 nm (ferulic acid, p-coumaric acid, vanillin, vanillic acid, p-hydroxybenzaldehyde) and 254 nm (p-hydroxybenzoic acid, protocatechuic acid) with a sample injection volume of 10 μl. The retention times of the samples were compared with those of purified standards (Sigma-Aldrich, U.S.A.) for identification and quantification.

### Gas Chromatography—Mass Spectrometry (GC-MS) Identification and Quantification

To extract organic acids (β-ketoadipic acid, adipic acid and levulinic acid) for detection, 500 μL of 1 M HCl, 300 μL of ethyl acetate and 100 μL of an internal standard (1,14-tetradecanedioic acid) were added to 1 mL of a cell culture sample. Subsequently, cells were disrupted by bead beating (FastPrep-24™ 5G and acid-washed beads (≤106 μm) running at 6.5 m/s and 1 min interval 4 times) and centrifuged for 10 min at 20,000×g at 4° C. to separate the organic phase. The ethyl acetate extracts were incubated with a derivatization agent (BSTFA with 1% trimethylchlorosilane (TCMS)) overnight prior to analysis by gas-liquid chromatography (GC) using an Agilent 7890B GC system equipped with an HP-5MS column (Agilent) coupled to a mass spectrometer (Agilent 5977).

### Example 2

### An Enzymatic Pathway Enabling OPEFB Lignin Utilization

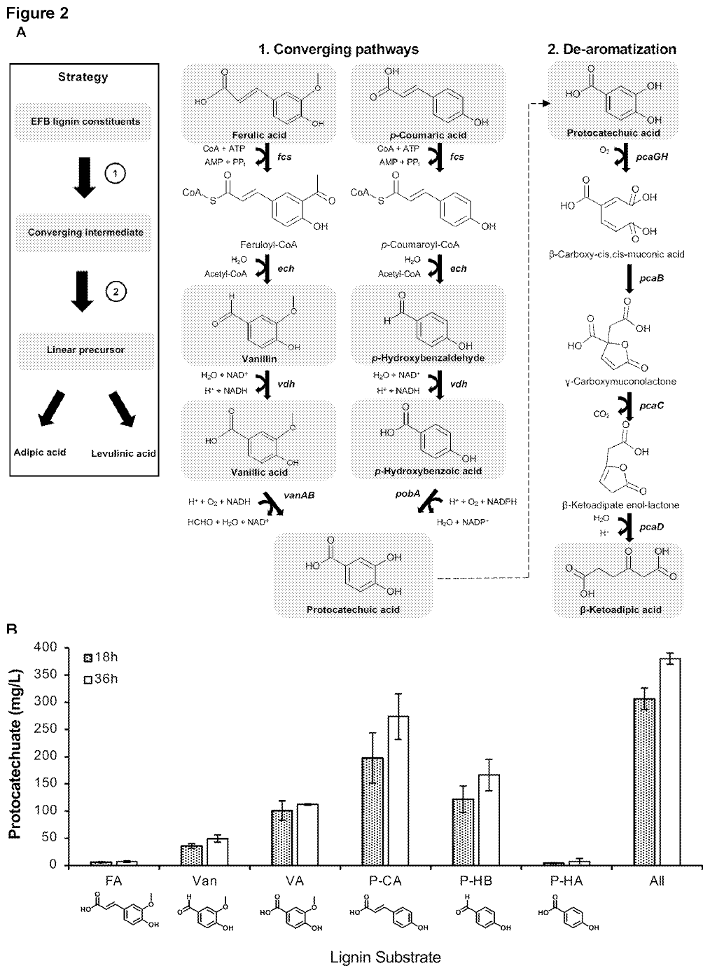

As a first step to convert the depolymerized OPEFB lignin to value-added chemicals, a 9-enzyme pathway (FIG. 2A, FIG. 3d) derived from Pseudomonas putida KT2440 was designed and assembled in Escherichia coli K-12 MG1655, a workhorse of industrial biotechnology. The ability of the E. coli K-12 MG1655 strain containing this metabolic pathway to utilize all OPEFB lignin derivatives, convert them into the single compound protocatechuic acid and subsequently convert that to a linear precursor, β-ketoadipic acid, was examined.

First, a converging pathway was constructed that comprises feruloyl-CoA synthetase (Fcs), enoyl-CoA hydratase (Ech), vanillin dehydrogenase (Vdh), vanillate O-demethylase (VanAB), and p-hydroxybenzoate hydroxylase (PobA) (FIG. 3C). To demonstrate the feasibility of the constructed pathway functioning in the microbial host, the bioconversion of individual lignin substrates for protocatechuic acid production was validated first (FIG. 2, Table 2), followed by bioconversion of OPEFB lignin derivatives. The results show that the converging pathway is able to utilize all of the OPEFB lignin derivatives, i.e., vanillic acid, ferulic acid, p-hydroxybenzaldehyde, p-coumaric acid, p-hydroxybenzoic acid and vanillin (in descending order of conversion efficiency), and convert them into the single intermediate molecule protocatechuic acid. The most efficient conversion was observed with vanillic acid and p-coumaric acid, producing ˜100% and ˜70% of theoretical yields, respectively (Table 2).

When the OPEFB lignin derivatives (formulated in the ratio that is naturally found after the pre-treatment) were tested, up to 400 mg/L protocatechuic acid was detected, reaching 11.5% of theoretical yield. The less-than-expected yield is mostly due to the inefficient utilization of vanillin, where only 2.7% molar conversion to protocatechuic acid was observed despite its high starting concentration (1.8 g/L). As a high concentration of vanillin has been reported to inhibit bacterial growth [Zaldivar et al., Biotechnology and Bioengineering 65: 24-33 (1999)], one possible approach to improve vanillin utilization would be to oxidize the depolymerized OPEFB lignin mixture, especially vanillin [Fargues et al., Chemical Engineering & Technology 19: 127-136 (1996)], to the less toxic compound vanillic acid prior to its feeding to the engineered cells. As vanillic acid has shown complete conversion, this approach may improve the yield of bioproduction and reduce toxicity to the host cells. However, these approaches have not been fully explored in this study, as the aim of this work is to first demonstrate the feasibility of direct conversion of the OPEFB lignin cocktail.

After the successful production of protocatechuic acid from the OPEFB lignin derivatives, a de-aromatization pathway involving protocatechuate 3,4-dioxygenase (PcaGH), 3-carboxy-cis,cis-muconate cycloisomerase (PcaB), 4-carboxymuconolactone decarboxylase (PcaC) and β-ketoadipate enol-lactone hydrolase (PcaD) (FIG. 2) was shown to function together with the organic acid production pathway in the subsequent experiment.

### Example 3

### De Novo Organic Acid Production Pathways from β-Ketoadipic Acid in E. coli

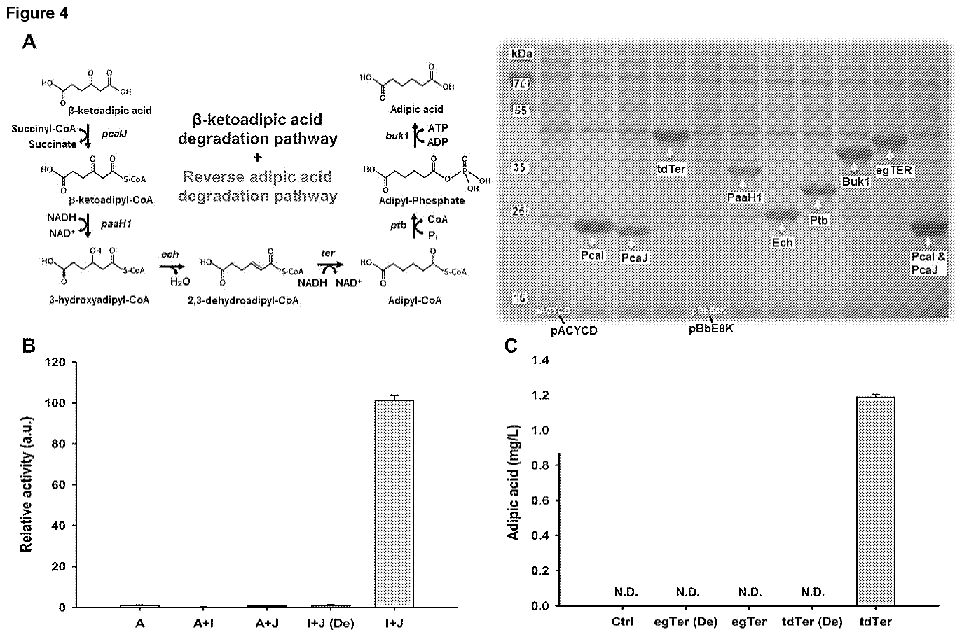

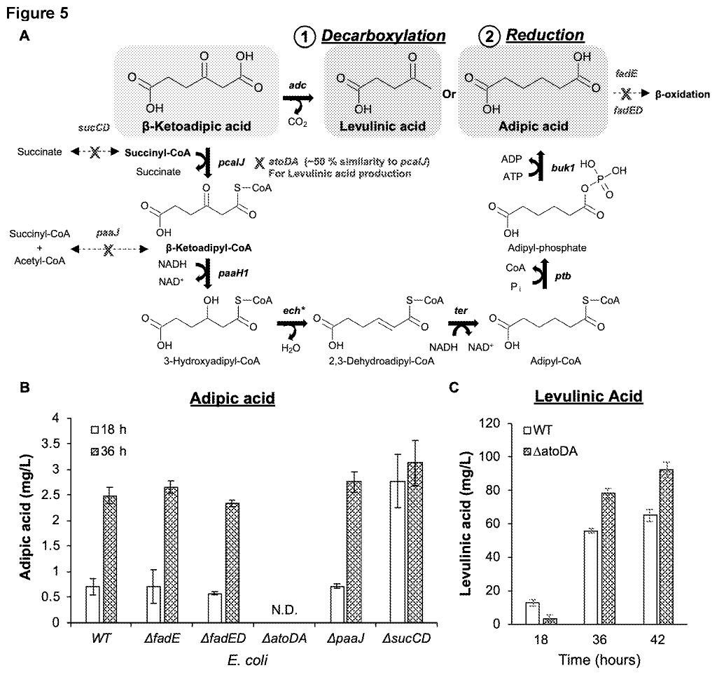

A direct biosynthesis of adipic acid from carbon sources in E. coli has been reported [Yu et al., Biotechnol Bioeng 111: 2580-2586 (2014); Cheong et al., Nat Biotechnol 34: 556-561 (2016); Zhao et al., Metabolic Engineering 47: 254-262 (2018)] where artificial adipic acid synthesis pathways were constructed to convert glucose or glycerol to adipic acid. In a recent study, Niu et al. [Niu et al., Metabolic Engineering 59: 151-161(2020)] successfully demonstrated adipic acid production from β-ketoadipic acid in Pseudomonas putida KT2440. Adapted from these findings, an adipic production pathway was constructed and validated (FIG. 4A) in E. coli. The constructed pathway utilizes β-ketoadipic acid by (1) esterifying it with CoA by β-ketoadipic acid succinyl-CoA transferase (PcaIJ); (2) subsequently reducing the 3-oxo group by 3-hydroxyacyl-CoA dehydrogenase (PaaH1), enoyl-CoA hydratase (Ech), and trans-enoyl-CoA reductase (Ter); and (3) removing CoA by phosphate butyryltransferase (Ptb) and butyrate kinase 1 (Buk1) to form adipic acid. To test this entire pathway, the 6 enzymes were first individually expressed in E. coli BL21(DE3) (FIG. 4A) and subsequently characterized for their activity. In vitro enzymatic activities for β-ketoadipic acid degradation and 3-oxo reduction to adipic acid (FIG. 4B, 4C) were measured. We observed that 3-oxo reduction required screening for a suitable reducing enzyme, Ter, that is responsible for converting 2,3-dehydroadipyl-CoA to adipyl-CoA. Adipic acid (1.18 mg/L) was detected only when Ter from Treponema denticola (TdTer) was used and not when the Ter from Euglena gracilis (EgTer) was used. Through this systematic in vitro enzymatic characterization, we verified the novel enzymatic pathway from β-ketoadipic acid to adipic acid (FIG. 5A).

Unlike the adipic acid pathway, levulinic acid production involves a single decarboxylation step from β-ketoadipic acid. This reaction was catalyzed by acetoacetate decarboxylase (Adc) from Clostridium acetobutylicum [Cheong et al., Nat Biotechnol 34: 556-561 (2016)]. The level of levulinic acid exceeded 60 mg/L within 36 h of bioconversion under shake-flask conditions (FIG. 5C).

### Example 4

### Host Engineering for Optimizing Value-Added Chemical Production

To facilitate the conversion of β-ketoadipic acid, repurposing of the existing native metabolic pathway in E. coli was required to channel reduction and decarboxylation pathways (FIG. 5A). This involved using the E. coli K-12 MG1655 reference genomic model in the EcoCyc database [Keseler et al., Nucleic Acids Research 45: D543-D550 (2016)] to search for potential native genes that may be able to divert intermediates or cofactors to other products. We postulated that native genes of E. coli may compete and negatively impact the designed pathways, namely, fadE, fadED (fadE; SEQ ID NO: 43 and fadD; SEQ ID NO: 45), paaJ (SEQ ID NO: 51), and sucCD (sucC; SEQ ID NO: 53 and sucD; SEQ ID NO: 55) for adipic acid production and atoDA (atoD; SEQ ID NO: 47 and atoA; SEQ ID NO: 49) for levulinic acid production. We assessed the impact of each gene deletion based on bioconversion of p-coumaric acid and used the amount produced as an indicator for pathway repurposing efficiency (FIG. 5B, C).

For adipic acid production, acyl-CoA dehydrogenase (fadE) and long-chain fatty acid CoA ligase (fadD) or both (fadED) genes were targeted, as these are involved in fatty acid metabolism, which can potential utilize six-carbon dicarboxylic acid (adipic acid) for β-oxidation [Lennen et al., Biotechnol Bioeng 106: 193-202 (2010); Sathesh-Prabu and Lee, J Agric Food Chem 63: 8199-8208 (2015)]. This metabolism can potentially utilize six-carbon dicarboxylic acid (adipic acid) for β-oxidation [Smit et al., Biotechnol Lett 27: 859-864 (2005)]. However, the deletion of these genes did not significantly improve adipic acid production. With the focus of channeling the flux towards β-ketoadipyl-CoA, AtoDA (AtoD; SEQ ID NO: 48 and AtoA; SEQ ID NO: 50) which shares ˜50% amino acid sequence similarity to the engineered PcaIJ (PcaI; SEQ ID NO: 26 and PcaJ; SEQ ID NO: 28) was inactivated. However, presence of the atoDA gene was found to play a pivotal role in initiating this new pathway, as deletion resulted in complete ablation of adipic acid production. As succinyl-CoA is an important cofactor in the formation of β-ketoadipyl-CoA, sucCD (sucC; SEQ ID NO: 53 and sucD; SEQ ID NO: 55, which encode subunits of succinyl-CoA synthetase) were deleted to minimize the competing conversion of succinyl-CoA towards succinate [Birney et al., J Bacteriol 178: 2883-2889 (1996); Zhao et al., Metabolic Engineering 47: 254-262 (2018)]. β-Ketoadipyl-CoA thiolase (paaJ; SEQ ID NO: 51) was also targeted for deletion due to its role in the reversible catalysis of β-ketoadipyl-CoA to succinyl-CoA and acetyl-CoA [Yu et al., Biotechnol Bioeng 111: 2580-2586 (2014); Babu et al., Process Biochemistry 50: 2066-2071 (2015)]. Among the list of genes that can potentially shunt intermediates from the introduced reduction reactions, sucCD deletion resulted in the greatest improvement in adipic acid production. The sucCD mutant was able to convert the substrates at an approximately 3-fold higher efficiency than other mutants, as shown by the higher production observed at an early time point (18 h) (FIG. 5B).

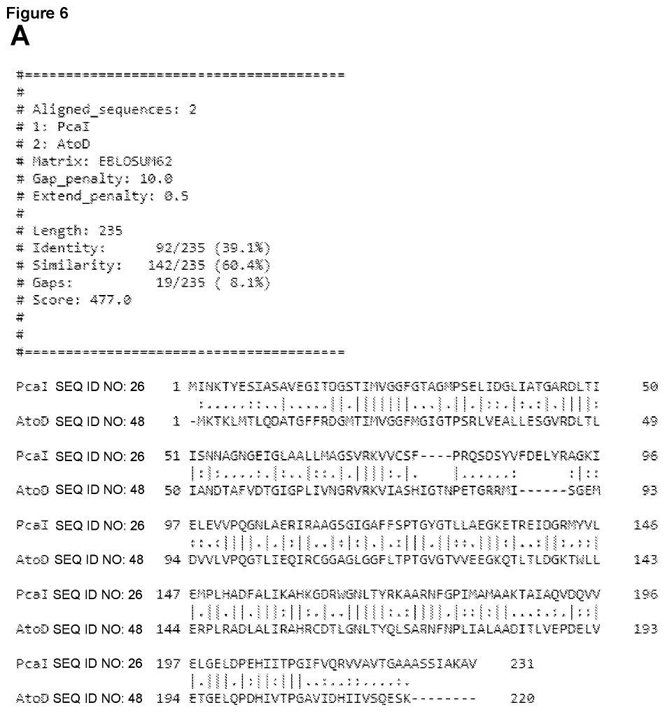

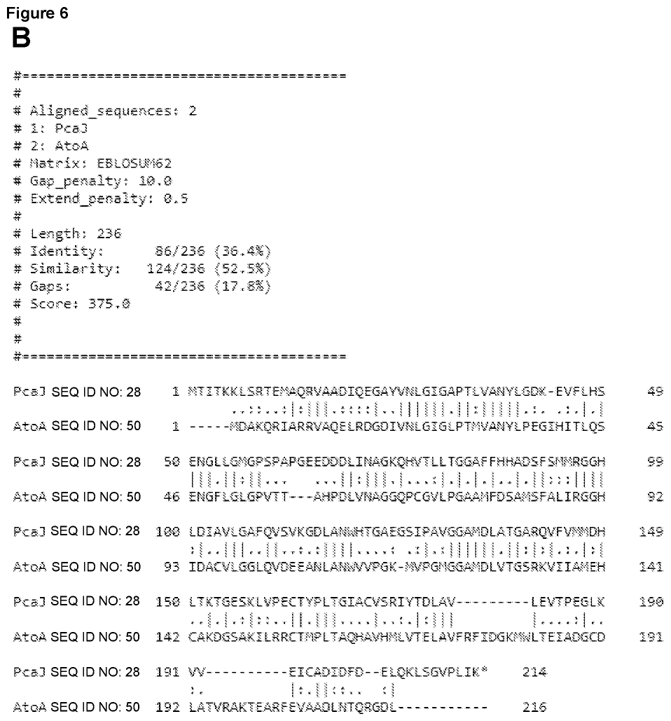

For levulinic acid production, as acetoacetate decarboxylase (Adc)[Cheong et al., Nat Biotechnol 34: 556-561 (2016)] was used to convert β-ketoadipic acid, the atoDA genes from the acetoacetic acid degradation pathway in E. coli were targeted for deletion. The atoDA genes were targeted because they share >50% amino acid sequence similarity with the pca/J-encoded enzyme based on sequence alignment (FIG. 6). Based on the similarity, the deletion was anticipated to promote the intended decarboxylation. Indeed, the atoDA-deleted strain was able to promote an approximately 40% increase in levulinic acid compared to its level in wild-type E. coli (FIG. 5C).

Overall, the outcome of the host engineering approaches indicates that the sucCD-deleted host is suitable for adipic acid production and that the atoDA-deleted host is suitable for levulinic acid production. These two strains were used in subsequent experiments for downstream optimization.

### Example 5

### Genetic Controller Enabling Autonomous OPEFB Lignin-Derived Hydroxycinnamic Acid-Dependent Regulation

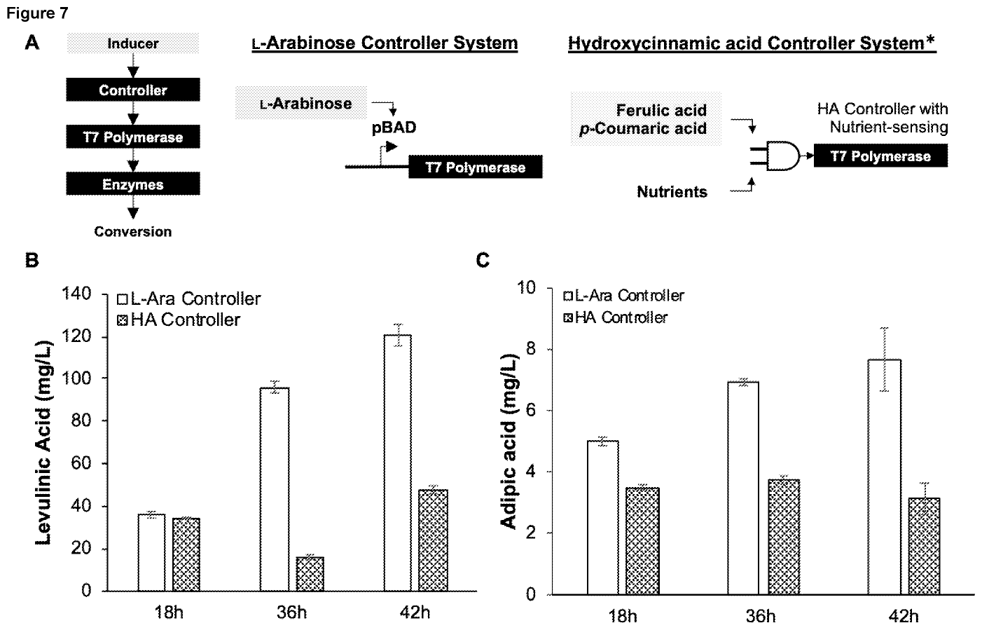

During pathway validation and host engineering experiments, an induction system based on the L-arabinose [Guzman et al., J Bacteriol 177: 4121-4130 (1995)] inducer was used to regulate the expression of pathway enzymes in a dose-dependent manner. The role of the genetic controller is to modulate downstream gene transcription through the expression of the bacteriophage-based T7 polymerase (FIG. 7A). Since the organic acid production pathways are long (15 enzymes for adipic acid production and 10 enzymes for levulinic acid), to ensure good transcription of all the genes, the genes were placed under the non-native, strong T7 promoter, which is recognized by the T7 polymerase to initiate downstream transcription. However, uncontrolled expression of the T7 polymerase can lead to overexpression of target proteins, which may burden the host cell [Kesik-Brodacka et al., Microbial Cell Factories 11: 109 (2012)]; hence, expression must be regulated by a genetic controller that restricts T7 polymerase transcription via external inputs such as chemical inducers.

Although the L-arabinose controller (pBAD) is an effective genetic device, it requires additional external resources, i.e., L-arabinose acts as an inducer, thus increasing the deployment cost of the biocatalytic cells. To improve the economics of OPEFB lignin utilization, we considered employing the hydroxycinnamic acid (HA) controller system reported by Lo et al., in 2016 [Lo et al., Cell Syst 3: 133-143 (2016)]. The HA controller system is inducible by HAs such as ferulic acid and p-coumaric acid, which are present in the depolymerized OPEFB lignin.

For comparison, we tested both L-arabinose (pBAD; SEQ ID NO: 41) and HA (SEQ ID NO: 42) controllers for adipic and levulinic acid production in optimized host strains (ΔsucCD and ΔatoDA, respectively) under shake-flask conditions at 30° C. and using p-coumaric acid (final concentration, 1 g/L) as the substrate (Table 1). The fermentation temperature was set at 30° C. instead of the commonly used 37° C. based on two reasons: 1) less energy is required to maintain a lower temperature while not impacting the growth of the engineered E. coli, and 2) the unstable compound β-ketoadipic acid can have a longer half-life for enzymatic conversion at the lower temperature. The L-arabinose-induced controller performed better than the HA controller in terms of the product yield: a 2-fold higher titer was observed for both adipic and levulinic acid production (FIG. 7B, C). We hypothesized that the concentration of the inducer L-arabinose (0.2% w/v) used in the shake-flask experiment resulted in rapid overexpression of enzymes in a given time frame, resulting in higher bioconversion than the HA controller.

### Example 6

### Optimized Host with Hydroxycinnamic Acid Controller for Efficient OPEFB Lignin Utilization and Bioconversion

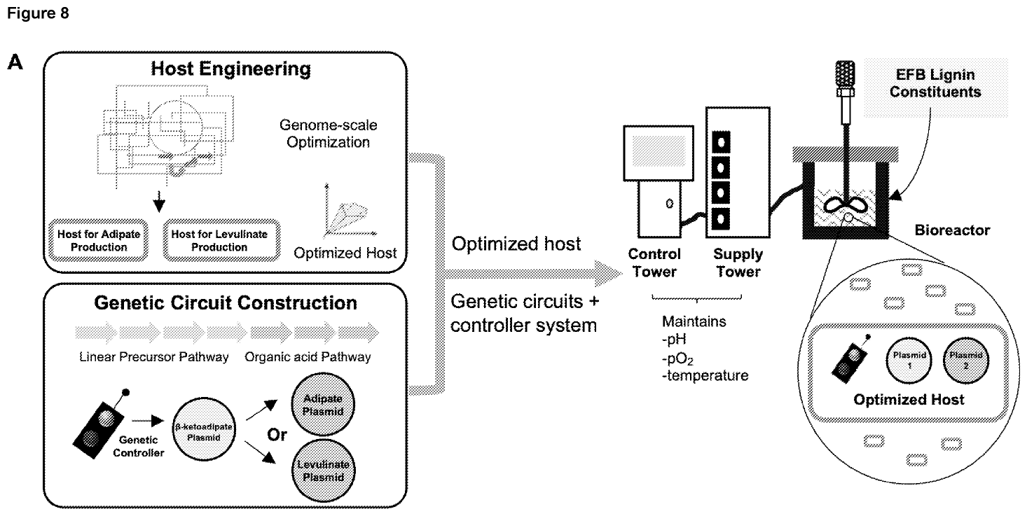

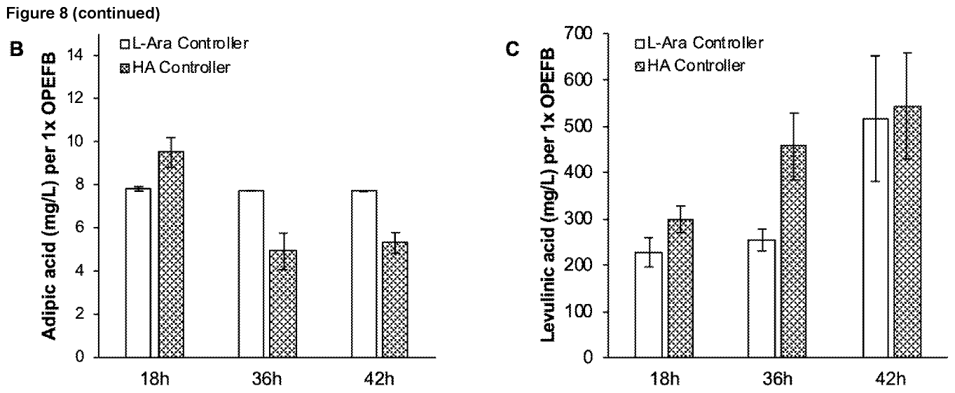

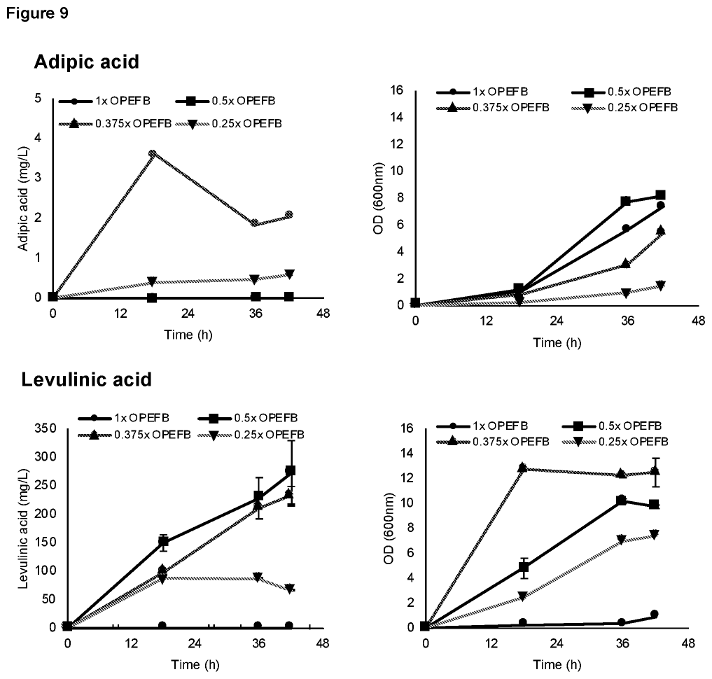

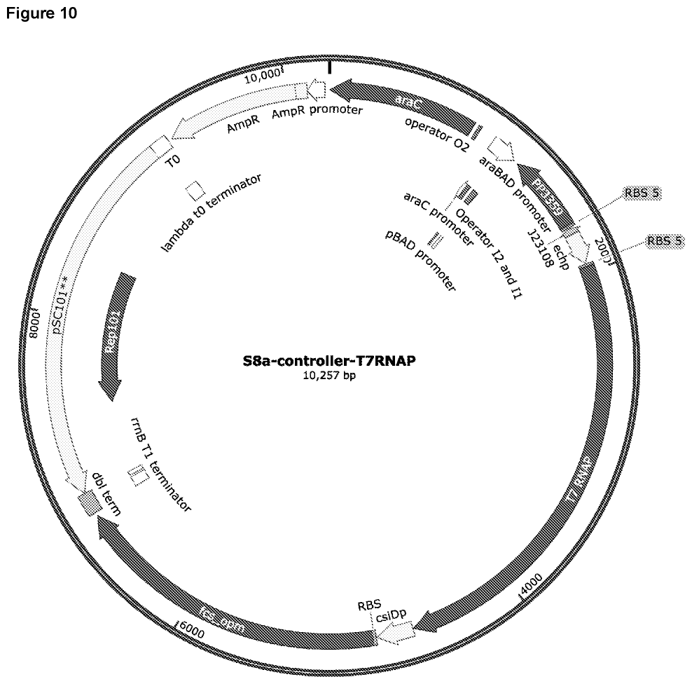

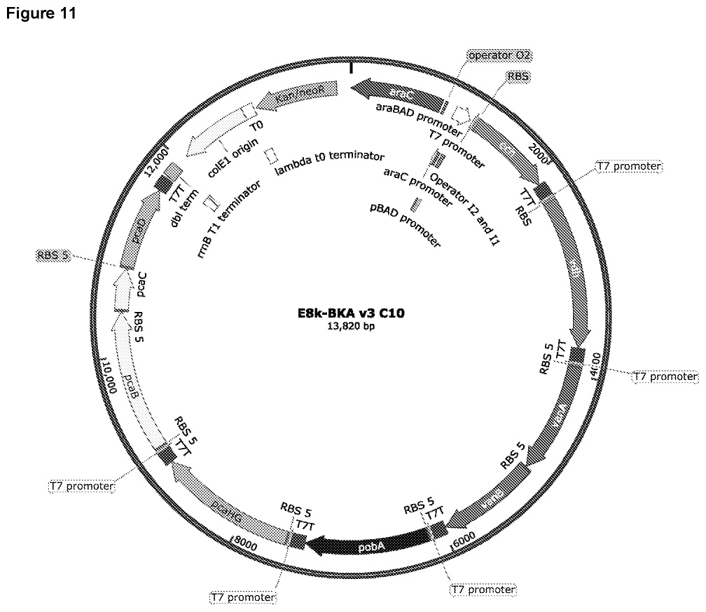

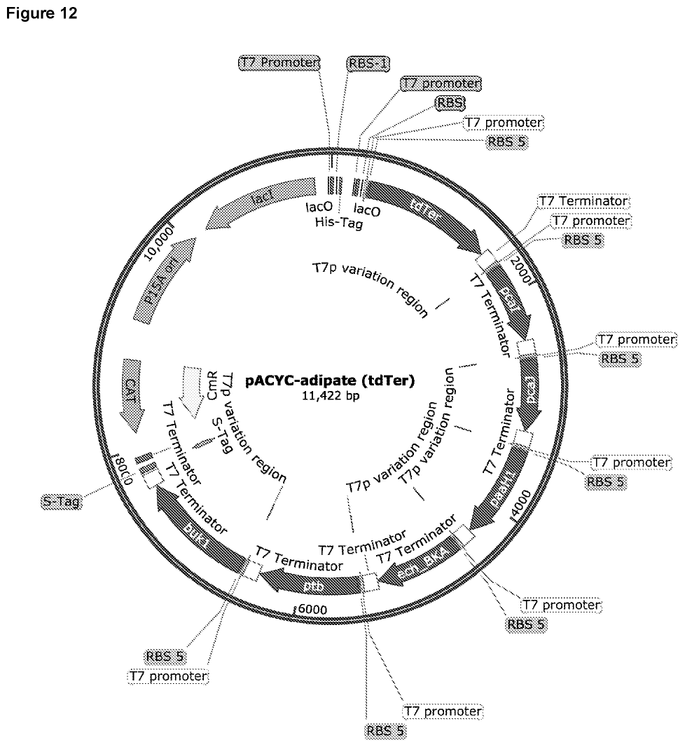

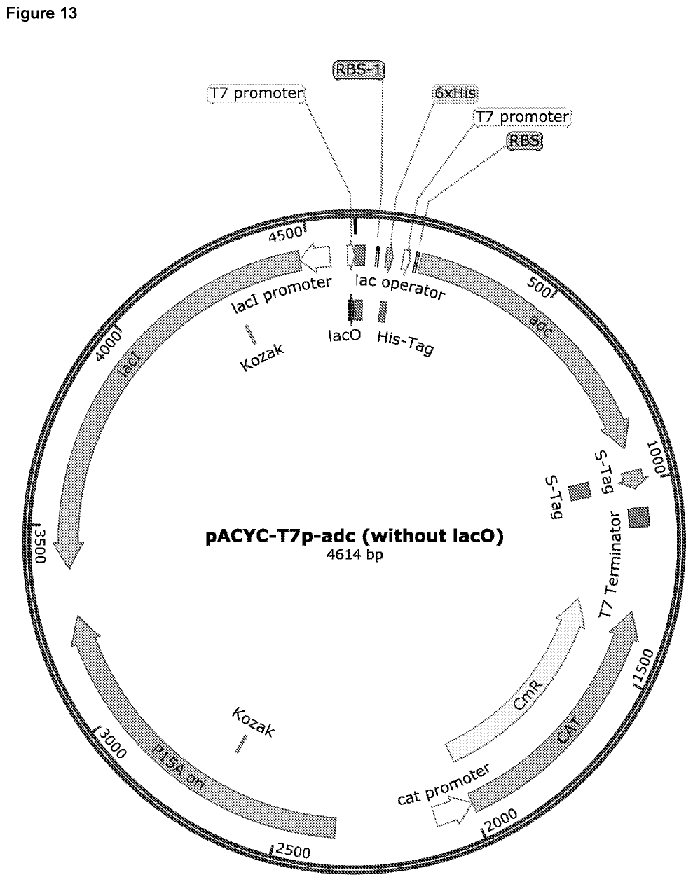

In an attempt to further boost the yield, the inherent problems faced by shake-flask experiments, such as limited oxygen levels and uncontrolled pH, which can affect the productivity of microbial hosts was overcome by using a controlled bioreactor which can regulate these parameters during the fermentation process. Bioreactors with oxygen (pO₂) and pH sensors and their relevant pumps to maintain these parameters at target values were used for OPEFB lignin conversion (FIG. 8A). The concerns for oxygen availability and the need for pH regulation are due to i) the need for oxygen for cellular metabolic growth and de-aromatization of protocatechuic acid and ii) CO₂ generation during adipic or levulinic acid production that leads to a decreasing pH in the cell culture over time. To further improve the bioconversion of the OPEFB lignin cocktail by the engineered cells, the feed dosage to the bioreactors was optimized (FIG. 9—0.5× OPEFB lignin for levulinic acid conversion and 0.375× OPEFB lignin for adipic acid conversion).

Under controlled conditions, the respective optimized host strains (ΔsucCD and ΔatoDA) bearing the HA controller performed similar to, if not slightly better, than the strains bearing the L-arabinose controller: ˜1.8-fold higher titer was observed for levulinic acid production (455.7 mg/L vs. 253.5 mg/L per 1×OPEFB lignin at 36 h) and ˜23% higher for adipic acid production (9.5 mg/L vs 7.8 mg/L per 1×OPEFB lignin at 18 h) in the HA controller strains (FIG. 8B). Given the modest production of adipic acid in our engineered cells, to identify a potential rate-limiting step(s) in our synthetic metabolic pathways (depicted in FIG. 2), we quantified the key substrates of the synthetic metabolic pathways in levulinic and adipic acid producing cells over time (FIG. 8C). This measurement shows a significant accumulation of vanillic aid in the adipic acid producing cells, suggesting that the enzymatic conversion of vanillic acid be a major rate-limiting step. In the levulinic acid producing cells, p-coumaric acid and ferulic acid were not fully utilized (FIG. 8C), which represent rate-limiting steps in the synthetic metabolic pathways. This result suggests that the production of levulinic acid and adipic acid in our engineered cells can be further increased if the aforementioned rate-limiting enzymatic reactions are improved.

### SUMMARY

Overall, this is the first report of autonomous cell-based production of adipic and levulinic acid from an OPEFB lignin cocktail without the need for upstream separation into individual derivatives before conversion and costly chemical inducers. Herein, we have demonstrated methods to produce adipic acid and levulinic acid from OPEFB lignin, primarily because both are industrially relevant chemicals that can be derived from the versatile dearomatized precursor β-ketoadipic acid.

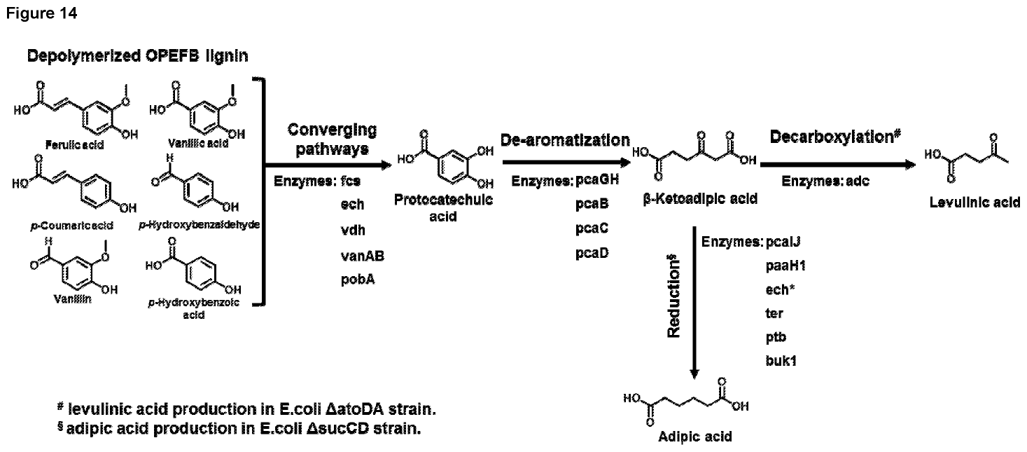

In this study, we demonstrate direct utilization of unfractionated depolymerized OPEFB lignin to produce commodity chemicals using an engineered E. coli strain. E. coli was engineered to have 3 genetic modules for the following functions: 1. genetic control for autonomous activation, 2. conversion of depolymerized lignin derivatives into β-ketoadipic acid by pathway enzymes, and 3. conversion of β-ketoadipic acid to commodity chemicals by pathway enzymes (FIG. 14). We demonstrated the production of adipic acid and levulinic acid using engineered E. coli, where up to 9.5 mg/L adipic acid and 455.57 mg/L levulinic acid were produced from reconstituted OPEFB lignin derivatives in fermenter-controlled conditions. The results of our research demonstrate a simple, one-pot biosynthesis approach that can potentially be used to directly utilize derivatives of agricultural waste to produce commodity chemicals.

### REFERENCES

Anderson, J. C., Dueber, J. E., Leguia, M., Wu, G. C., Goler, J. A., Arkin, A. P., and Keasling, J. D. (2010). BglBricks: A flexible standard for biological part assembly. Journal of Biological Engineering 4: 1.
Babu, T., Yun, E. J., Kim, S., Kim, D. H., Liu, K. H., Kim, S. R., and Kim, K. H. (2015). Engineering Escherichia coli for the production of adipic acid through the reversed β-oxidation pathway. Process Biochemistry 50: 2066-2071.
Barghini, P., Di Gioia, D., Fava, F., and Ruzzi, M. (2007). Vanillin production using metabolically engineered Escherichia coli under non-growing conditions. Microbial Cell Factories 6: 13.
Birney, M., Um, H. D., and Klein, C. (1996). Novel mechanisms of Escherichia coli succinyl-coenzyme A synthetase regulation. J Bacteriol 178: 2883-2889.
Cheong, S., Clomburg, J. M., and Gonzalez, R. (2016). Energy- and carbon-efficient synthesis of functionalized small molecules in bacteria using non-decarboxylative Claisen condensation reactions. Nat Biotechnol 34: 556-561.
Coral Medina, J. D., Woiciechowski, A., Zandona Filho, A., Noseda, M. D., Kaur, B. S., and Soccol, C. R. (2015). Lignin preparation from oil palm empty fruit bunches by sequential acid/alkaline treatment—A biorefinery approach. Bioresource Technology 194: 172-178.
Datsenko, K. A., and Wanner, B. L. (2000). One-step inactivation of chromosomal genes in Escherichia coli K-12 using PCR products. Proc Natl Acad Sci USA 97: 6640-6645.
Deng, Y., Ma, L., and Mao, Y. (2016). Biological production of adipic acid from renewable substrates: Current and future methods. Biochemical Engineering Journal 105: 16-26.
Fargues, C., Mathias, Á., Silva, J., and Rodrigues, A. (1996). Kinetics of vanillin oxidation. Chemical Engineering & Technology 19: 127-136.
Ghorpade, V., and Hanna, M. (1997). “Industrial Applications for Levulinic Acid,” in Cereals: Novel Uses and Processes, eds. G. M. Campbell, C. Webb & S. L. Mckee. (Boston, Mass.: Springer US), 49-55.
Guzman, L. M., Belin, D., Carson, M. J., and Beckwith, J. (1995). Tight regulation, modulation, and high-level expression by vectors containing the arabinose PBAD promoter. J Bacteriol 177: 4121-4130.
Heckman, K. L., and Pease, L. R. (2007). Gene splicing and mutagenesis by PCR-driven overlap extension. Nature Protocols 2: 924-932.
Keseler, I. M., Mackie, A., Santos-Zavaleta, A., Billington, R., Bonavides-Martinez, C., Caspi, R., Fulcher, C., Gama-Castro, S., Kothari, A., Krummenacker, M., Latendresse, M., Muñiz-Rascado, L., Ong, Q., Paley, S., Peralta-Gil, M., Subhraveti, P., Velázquez-Ramirez, D. A., Weaver, D., Collado-Vides, J., Paulsen, I., and Karp, P. D. (2016). The EcoCyc database: reflecting new knowledge about Escherichia coli K-12. Nucleic Acids Research 45: D543-D550.
Kesik-Brodacka, M., Romanik, A., Mikiewicz-Sygula, D., Plucienniczak, G., and Plucienniczak, A. (2012). A novel system for stable, high-level expression from the T7 promoter. Microbial Cell Factories 11, 109.
Lee, T., Krupa, R., Zhang, F., Hajimorad, M., Holtz, W., Prasad, N., Lee, S., and Keasling, J. (2011). BgIBrick vectors and datasheets: A synthetic biology platform for gene expression. Journal of Biological Engineering 5: 12.
Lennen, R. M., Braden, D. J., West, R. A., Dumesic, J. A., and Pfleger, B. F. (2010). A process for microbial hydrocarbon synthesis: Overproduction of fatty acids in Escherichia coli and catalytic conversion to alkanes. Biotechnol Bioeng 106: 193-202.
Li, Q., Ng, W. T., Puah, S. M., Bhaskar, R. V., Soh, L. S., Macbeath, C., Parakattil, P., Green, P., and Wu, J. C. (2014). Efficient production of fermentable sugars from oil palm empty fruit bunch by combined use of acid and whole cell culture-catalyzed hydrolyses. Biotechnology and Applied Biochemistry 61: 426-431.
Lo, T. M., Chng, S. H., Teo, W. S., Cho, H. S., and Chang, M. W. (2016). A Two-Layer Gene Circuit for Decoupling Cell Growth from Metabolite Production. Cell Syst 3: 133-143.
Maclean, A. M., Macpherson, G., Aneja, P., and Finan, T. M. (2006). Characterization of the beta-ketoadipate pathway in Sinorhizobium meliloti. Appl Environ Microbiol 72: 5403-5413.
Marketwatch (2020). Levulinic Acid Market Size 2020: Top Countries Data, Defination, Detailed Analysis of Current Industry Figures with Forecasts Growth By 2026 [Online]. Available: worldwidewebdotmarketwatchdotcom/press-release/levulinic-acid-market-size-2020-top-countries-data-defination-detailed-analysis-of-current-industry-figures-with-forecasts-growth-by-2026-2020-07-13 [Accessed].
Mohamad Ibrahim, M. N., Nadiah, M. Y. N., Norliyana, M. S., Sipaut, C. S., and Shuib, S. (2008). Separation of Vanillin from Oil Palm Empty Fruit Bunch Lignin. CLEAN—Soil, Air, Water 36: 287-291.
Murphy, D. (2014). The future of oil palm as a major global crop: Opportunities and challenges. Journal of Oil Palm Research 26: 1-24.
Niu, W., Willett, H., Mueller, J., He, X., Kramer, L., Ma, B., and Guo, J. (2020). Direct biosynthesis of adipic acid from lignin-derived aromatics using engineered Pseudomonas putida KT2440. Metabolic Engineering. 59: 151-161
Polen, T., Spelberg, M., and Bott, M. (2013). Toward biotechnological production of adipic acid and precursors from biorenewables. J Biotechnol 167: 75-84.
Research, G. V. (2018). Adipic Acid Market Size, Share & Trends Analysis Report By Application (Nylon 66 Fiber, Nylon 66 Resin, Polyurethane, Adipate Ester), By Region (APAC, North America, Europe, MEA, CSA), And Segment Forecasts, 2018-2024 [Online]. Available: worldwidewebdotgrandviewresearchdotcom/industry-analysis/adipic-acid-market [Accessed].
Sathesh-Prabu, C., and Lee, S. K. (2015). Production of Long-Chain alpha, omega-Dicarboxylic Acids by Engineered Escherichia coli from Renewable Fatty Acids and Plant Oils. J Agric Food Chem 63: 8199-8208.
Smit, M. S., Mokgoro, M. M., Setati, E., and Nicaud, J. M. (2005). alpha, omega-Dicarboxylic acid accumulation by acyl-CoA oxidase deficient mutants of Yarrowia lipolytica. Biotechnol Lett 27: 859-864.
Vardon, D. R., Franden, M. A., Johnson, C. W., Karp, E. M., Guarnieri, M. T., Linger, J. G., Salm, M. J., Strathmann, T. J., and Beckham, G. T. (2015). Adipic acid production from lignin. Energy & Environmental Science 8: 617-628.
Wells, T., Jr., and Ragauskas, A. J. (2012). Biotechnological opportunities with the beta-ketoadipate pathway. Trends Biotechnol 30, 627-637.
Xu, W., Miller, S. J., Agrawal, P. K., and Jones, C. W. (2012). Depolymerization and Hydrodeoxygenation of Switchgrass Lignin with Formic Acid. ChemSusChem 5: 667-675.
Yu, J. L., Xia, X. X., Zhong, J. J., and Qian, Z. G. (2014). Direct biosynthesis of adipic acid from a synthetic pathway in recombinant Escherichia coli. Biotechnol Bioeng 111: 2580-2586.
Zaldivar, J., Martinez, A., and Ingram, L. O. (1999). Effect of selected aldehydes on the growth and fermentation of ethanologenic Escherichia coli. Biotechnology and Bioengineering 65: 24-33.
Zhao, M., Huang, D., Zhang, X., Koffas, M.a.G., Zhou, J., and Deng, Y. (2018). Metabolic engineering of Escherichia coli for producing adipic acid through the reverse adipate-degradation pathway. Metabolic Engineering 47: 254-262.

1. An isolated genetically engineered microorganism for producing β-ketoadipic acid from depolymerized lignin, wherein the microorganism has been transformed by at least one polynucleotide molecule; the at least one polynucleotide molecule comprising heterologous β-ketoadipic acid pathway genes, feruloyl-CoA synthetase (fcs), enoyl-CoA hydratase (ech), vanillin dehydrogenase (vdh), vanillate O-demethylase (vanAB; vanA and vanB), p-hydroxybenzoate hydroxylase (pobA), protocatechuate 3,4-dioxygenase (pcaGH; pcaG and pcaH), 3-carboxy-cis,cis-muconate cycloisomerase (pcaB), 4-carboxymuconolactone decarboxylase (pcaC) and β-ketoadipate enol-lactone hydrolase (pcaD) operably linked to at least one promoter, wherein said genetically engineered microorganism can convert depolymerized lignin to β-ketoadipic acid.

2. The isolated genetically engineered microorganism according to claim 1, further comprising:
(a) heterologous β-ketoadipic acid utilization genes, β-ketoadipic acid succinyl-CoA transferase (pcaIJ; pcaI and pcaJ), 3-hydroxyacyl-CoA dehydrogenase (paaH1), enoyl-CoA hydratase (ech), trans-enoyl-CoA reductase (ter), phosphate butyryltransferase (ptb) and butyrate kinase 1 (buk1) operably linked to at least one promoter, wherein said genetically engineered microorganism can convert 3-ketoadipic acid to adipic acid, and/or
(b) a heterologous β-ketoadipic acid utilization gene, acetoacetate decarboxylase (adc), operably linked to at least one promoter, wherein said genetically engineered microorganism can convert β-ketoadipic acid to levulinic acid.

3. The isolated genetically engineered microorganism according to claim 1, wherein:
the fcs gene encodes an amino acid sequence set forth in SEQ ID NO: 2; and/or
the ech gene encodes an amino acid sequence set forth in SEQ ID NO: 4; and/or
the vdh gene encodes an amino acid sequence set forth in SEQ ID NO: 6; and/or
the vanA gene encodes an amino acid sequence set forth in SEQ ID NO: 8; and/or
the vanB gene encodes an amino acid sequence set forth in SEQ ID NO: 10; and/or
the pobA gene encodes an amino acid sequence set forth in SEQ ID NO: 12; and/or
the pcaH gene encodes an amino acid sequence set forth in SEQ ID NO: 14; and/or
the pcaG gene encodes an amino acid sequence set forth in SEQ ID NO: 16; and/or
the pcaB gene encodes an amino acid sequence set forth in SEQ ID NO: 18; and/or
the pcaC gene encodes an amino acid sequence set forth in SEQ ID NO: 20; and/or
the pcaD gene encodes an amino acid sequence set forth in SEQ ID NO: 22; and/or
the ter gene encodes an amino acid sequence set forth in SEQ ID NO: 24; and/or
the pcaI gene encodes an amino acid sequence set forth in SEQ ID NO: 26; and/or
the pcaJ gene encodes an amino acid sequence set forth in SEQ ID NO: 28; and/or
the paaH1 gene encodes an amino acid sequence set forth in SEQ ID NO: 30; and/or
the ech gene encodes an amino acid sequence set forth in SEQ ID NO: 32; and/or
the ptb gene encodes an amino acid sequence set forth in SEQ ID NO: 34; and/or
the buk1 gene encodes an amino acid sequence set forth in SEQ ID NO: 36; and/or
the adc gene encodes an amino acid sequence set forth in SEQ ID NO: 38.

4. The isolated genetically engineered microorganism according to claim 3, wherein:
the fcs gene comprises a nucleic acid sequence at least 80% sequence identity to the polynucleotide sequence set forth in SEQ ID NO: 1; and/or
the ech gene comprises a nucleic acid sequence at least 80% sequence identity to the polynucleotide sequence set forth in SEQ ID NO: 3; and/or
the vdh gene comprises a nucleic acid sequence at least 80% sequence identity to the polynucleotide sequence set forth in SEQ ID NO: 5; and/or
the vanA gene comprises a nucleic acid sequence at least 80% sequence identity to the polynucleotide sequence set forth in SEQ ID NO: 7; and/or
the vanB gene comprises a nucleic acid sequence at least 80% sequence identity to the polynucleotide sequence set forth in SEQ ID NO: 9; and/or
the pobA gene comprises a nucleic acid sequence at least 80% sequence identity to the polynucleotide sequence set forth in SEQ ID NO: 11; and/or
the pcaH gene comprises a nucleic acid sequence at least 80% sequence identity to the polynucleotide sequence set forth in SEQ ID NO: 13; and/or
the pcaG gene comprises a nucleic acid sequence at least 80% sequence identity to the polynucleotide sequence set forth in SEQ ID NO: 15; and/or
the pcaB gene comprises a nucleic acid sequence at least 80% sequence identity to the polynucleotide sequence set forth in SEQ ID NO: 17; and/or
the pcaC gene comprises a nucleic acid sequence at least 80% sequence identity to the polynucleotide sequence set forth in SEQ ID NO: 19; and/or
the pcaD gene comprises a nucleic acid sequence at least 80% sequence identity to the polynucleotide sequence set forth in SEQ ID NO: 21; and/or
the ter gene comprises a nucleic acid sequence at least 80% sequence identity to the polynucleotide sequence set forth in SEQ ID NO: 23; and/or
the pcaI gene comprises a nucleic acid sequence at least 80% sequence identity to the polynucleotide sequence set forth in SEQ ID NO: 25; and/or
the pcaJ gene comprises a nucleic acid sequence at least 80% sequence identity to the polynucleotide sequence set forth in SEQ ID NO: 27; and/or
the paaH1 gene comprises a nucleic acid sequence at least 80% sequence identity to the polynucleotide sequence set forth in SEQ ID NO: 29; and/or
the ech gene comprises a nucleic acid sequence at least 80% sequence identity to the polynucleotide sequence set forth in SEQ ID NO: 31; and/or
the ptb gene comprises a nucleic acid sequence at least 80% sequence identity to the polynucleotide sequence set forth in SEQ ID NO: 33; and/or
the buk1 gene comprises a nucleic acid sequence at least 80% sequence identity to the polynucleotide sequence set forth in SEQ ID NO: 35; and/or
the adc gene comprises a nucleic acid sequence at least 80% sequence identity to the polynucleotide sequence set forth in SEQ ID NO: 37.

5. The isolated genetically engineered microorganism according to claim 1, wherein the at least one promoter is modulated by a heterologous genetic controller and/or is a constitutive promoter, such as T7.

6. (canceled)

7. The isolated genetically engineered microorganism according to claim 5, wherein the heterologous genetic controller is pBAD or hydroxycinnamic acid (HA).

8. The isolated genetically engineered microorganism according to claim 7, wherein the heterologous genetic controller pBAD comprises a nucleic acid sequence set forth in SEQ ID NO: 41 or the hydroxycinnamic acid (HA) controller comprises a nucleic acid sequence set forth in SEQ ID NO: 42.

9. The isolated genetically engineered microorganism according to claim 1, further comprising an inactivated endogenous succinyl CoA synthetase gene, such as sucCD and/or an inactivated β-ketoadipyl-CoA thiolase gene, such as paaJ.

10. The isolated genetically engineered microorganism according to claim 9, wherein the sucCD genes encode an amino acid sequence set forth in SEQ ID NO: 54 and SEQ ID NO: 56, respectively, and/or the paaJ gene encodes an amino acid sequence set forth in SEQ ID NO: 52.

11. The isolated genetically engineered microorganism according to claim 1, further comprising an inactivated endogenous Acyl CoA:acetate/3-ketoacid CoA transferase gene, such as atoDA.

12. The isolated genetically engineered microorganism according to claim 11, wherein the atoDA genes encode an amino acid sequence set forth in SEQ ID NO: 48 and SEQ ID NO: 50, respectively.

13. The isolated genetically engineered microorganism according to claim 1, comprising a bacteria or yeast, preferably a bacteria such as Escherichia coli.

14. The isolated genetically engineered microorganism according to claim 13, wherein the bacteria comprises Escherichia coli MG1655.

15. The isolated engineered microorganism according to claim 1, wherein the depolymerized lignin is from fibrous oil palm empty fruit bunches.

16. (canceled)

17. A recombinant vector comprising heterologous β-ketoadipic acid pathway genes, fcs, ech, vdh, vanAB (vanA and vanB), pobA, pcaGH (pcaG and pcaH), pcaB, pcaC and pcaD operably linked to at least one promoter, and/or
heterologous β-ketoadipic acid utilization genes, pcaIJ (pcaI and pcaJ), paaH1, ech, ter, ptb and buk1 operably linked to at least one promoter, and/or
a heterologous β-ketoadipic acid utilization gene, adc, operably linked to at least one promoter.

18. The recombinant vector of claim 17, wherein:
the fcs gene encodes an amino acid sequence set forth in SEQ ID NO: 2; and/or
the ech gene encodes an amino acid sequence set forth in SEQ ID NO: 4; and/or
the vdh gene encodes an amino acid sequence set forth in SEQ ID NO: 6; and/or
the vanA gene encodes an amino acid sequence set forth in SEQ ID NO: 8; and/or
the vanB gene encodes an amino acid sequence set forth in SEQ ID NO: 10; and/or
the pobA gene encodes an amino acid sequence set forth in SEQ ID NO: 12; and/or
the pcaH gene encodes an amino acid sequence set forth in SEQ ID NO: 14; and/or
the pcaG gene encodes an amino acid sequence set forth in SEQ ID NO: 16; and/or
the pcaB gene encodes an amino acid sequence set forth in SEQ ID NO: 18; and/or
the pcaC gene encodes an amino acid sequence set forth in SEQ ID NO: 20; and/or
the pcaD gene encodes an amino acid sequence set forth in SEQ ID NO: 22; and/or
the ter gene encodes an amino acid sequence set forth in SEQ ID NO: 24; and/or
the pcaI gene encodes an amino acid sequence set forth in SEQ ID NO: 26; and/or
the pcaJ gene encodes an amino acid sequence set forth in SEQ ID NO: 28; and/or
the paaH1 gene encodes an amino acid sequence set forth in SEQ ID NO: 30; and/or
the ech gene encodes an amino acid sequence set forth in SEQ ID NO: 32; and/or
the ptb gene encodes an amino acid sequence set forth in SEQ ID NO: 34; and/or
the buk1 gene encodes an amino acid sequence set forth in SEQ ID NO: 36; and/or
the adc gene encodes an amino acid sequence set forth in SEQ ID NO: 38.

19. The recombinant vector of claim 18, wherein:
the fcs gene comprises a nucleic acid sequence at least 80% sequence identity to the polynucleotide sequence set forth in SEQ ID NO: 1; and/or
the ech gene comprises a nucleic acid sequence at least 80% sequence identity to the polynucleotide sequence set forth in SEQ ID NO: 3; and/or
the vdh gene comprises a nucleic acid sequence at least 80% sequence identity to the polynucleotide sequence set forth in SEQ ID NO: 5; and/or
the vanA gene comprises a nucleic acid sequence at least 80% sequence identity to the polynucleotide sequence set forth in SEQ ID NO: 7; and/or
the vanB gene comprises a nucleic acid sequence at least 80% sequence identity to the polynucleotide sequence set forth in SEQ ID NO: 9; and/or
the pobA gene comprises a nucleic acid sequence at least 80% sequence identity to the polynucleotide sequence set forth in SEQ ID NO: 11; and/or
the pcaH gene comprises a nucleic acid sequence at least 80% sequence identity to the polynucleotide sequence set forth in SEQ ID NO: 13; and/or
the pcaG gene comprises a nucleic acid sequence at least 80% sequence identity to the polynucleotide sequence set forth in SEQ ID NO: 15; and/or
the pcaB gene comprises a nucleic acid sequence at least 80% sequence identity to the polynucleotide sequence set forth in SEQ ID NO: 17; and/or
the pcaC gene comprises a nucleic acid sequence at least 80% sequence identity to the polynucleotide sequence set forth in SEQ ID NO: 19; and/or
the pcaD gene comprises a nucleic acid sequence at least 80% sequence identity to the polynucleotide sequence set forth in SEQ ID NO: 21; and/or
the ter gene comprises a nucleic acid sequence at least 80% sequence identity to the polynucleotide sequence set forth in SEQ ID NO: 23; and/or
the pcaI gene comprises a nucleic acid sequence at least 80% sequence identity to the polynucleotide sequence set forth in SEQ ID NO: 25; and/or
the pcaJ gene comprises a nucleic acid sequence at least 80% sequence identity to the polynucleotide sequence set forth in SEQ ID NO: 27; and/or
the paaH1 gene comprises a nucleic acid sequence at least 80% sequence identity to the polynucleotide sequence set forth in SEQ ID NO: 29; and/or
the ech gene comprises a nucleic acid sequence at least 80% sequence identity to the polynucleotide sequence set forth in SEQ ID NO: 31; and/or
the ptb gene comprises a nucleic acid sequence at least 80% sequence identity to the polynucleotide sequence set forth in SEQ ID NO: 33; and/or
the buk1 gene comprises a nucleic acid sequence at least 80% sequence identity to the polynucleotide sequence set forth in SEQ ID NO: 35; and/or
the adc gene comprises a nucleic acid sequence at least 80% sequence identity to the polynucleotide sequence set forth in SEQ ID NO: 37.

20. A kit comprising an isolated genetically engineered microorganism according to claim 1.

21. A method of producing β-ketoadipic acid, adipic acid, or levulinic acid from depolymerized lignin, comprising the step of culturing a plurality of genetically engineered microorganisms of claim 1 under conditions for production of said β-ketoadipic acid, adipic acid, or levulinic acid, respectively.

22. (canceled)

23. (canceled)

24. The method of claim 21, further comprising isolating said product produced by the genetically engineered microorganisms, and/or
wherein the microorganism is a bacterium, such as Escherichia coli, preferably Escherichia coli MG1655, and/or
wherein the depolymerized lignin is from fibrous oil palm empty fruit bunches.

25. (canceled)

26. (canceled)
